(19) United States
(12) Patent Application Publication
(10) Pub. No.: US 2011/0274723 A1
(43) Pub. Date: Nov. 10, 2011

(54) CANCER IMMUNOTHERAPY AND METHOD OF TREATMENT

(75) Inventors: Adrian Ion Bot, Valencia, CA (US); Zhiyong Qiu, San Gabriel, CA (US); David C. Diamond, West Hills, CA (US); Mihail Obrocea, Santa Clarita, CA (US)

(73) Assignee: MANNKIND CORPORATION, Valencia, CA (US)

(21) Appl. No.: 12/910,778

(22) Filed: Oct. 22, 2010

Related U.S. Application Data
(60) Provisional application No. 61/279,621, filed on Oct. 23, 2009.
(60) Provisional application No. 61/254,657, filed on Oct. 24, 2009.
(60) Provisional application No. 61/255,850, filed on Oct. 28, 2009.

(51) Int. Cl. A61K 39/00; A61P 37/04; A61P 35/00

(52) U.S. Cl. 424/277.1

(57) ABSTRACT

The instant disclosure relates to methods for treating a cancer patient with an active immunotherapeutic, wherein the methods include assessing a patient's pre-existing immunoreactivity to at least one target antigen, choosing an immunotherapeutic regimen based on the level of pre-existing immunoreactivity, and administering an active immunotherapeutic according to the regimen.

### CROSS REFERENCE TO RELATED APPLICATIONS

This application claims priority under 35 U.S.C. § 119(e) from U.S. Provisional Applications Ser. Nos. 61/279,621 filed Oct. 23, 2009; 61/254,657 filed Oct. 24, 2009; and 61/255,850 filed Oct. 28, 2009, respectively; each of which is hereby incorporated by reference in its entirety.

### INCORPORATION OF INFORMATION PROVIDED IN ELECTRONIC FORMAT

The present application is being filed along with a Sequence Listing in electronic format. The Sequence Listing is provided as a file entitled 2010-10-22-SEQLISTING-0088480-062US0, created Oct. 22, 2010, which is 22 Kb in size. The information in the electronic format of the Sequence Listing is incorporated herein by reference in its entirety.

### FIELD OF THE INVENTION

The instant disclosure relates to strategies for the design and practice of immunotherapy protocols for generating an effector T cell response against a target antigen in a subject, and for more advantageously utilizing immunogenic products that induce, promote the growth of, activate, and/or otherwise stimulate effector T cells targeting tumor antigens in the treatment of cancer. More particularly, embodiments relate to determining a course of treatment and methods for treating a subject wherein the presence or absence of the baseline immunoreactivity to at least one target antigen is assessed to select subjects for administration of one or more active immunotherapeutics targeting the at least one target antigen. In some embodiments, the strategies disclosed herein include selecting patients with primarily lymphatically confined disease or disease that has limited progression beyond, or is otherwise confined to, the lymphatic system for treatment with an active immunotherapeutic that is administered intralymphatically.

### BACKGROUND

The American Cancer Society has estimated that almost 1.5 million new cases of cancer will be diagnosed this year (excluding carcinoma in situ of any site except urinary bladder and excluding basal and squamous cell skin cancers) and that approximately one out of every two American men and one out of every three American women will have some type of cancer at some point during their lifetime.

Normal body cells grow, divide, and die in an orderly fashion. In cell proliferative diseases such as cancer, cells, instead of dying, continue to grow out of control and divide. Although there are many kinds of cancer, the disease usually commences due to out-of-control growth of abnormal cells.

Usual treatment options for cancer include surgery, radiation therapy, and chemotherapy. An additional branch of treatment, which is referred to as immunotherapy, has more recently become established. Immunotherapies are designed to help the immune system recognize cancer cells, and/or to strengthen a response against cancer cells in order to destroy the cancer. Immunotherapies include active and passive immunotherapies. Active immunotherapies attempt to stimulate the body's own immune system to fight the disease. Passive immunotherapies generally do not rely on the patient's immune system to attack the disease; instead, they use immune system components (such as antibodies) generated outside of the patient's body.

Despite various types of cancer treatments, a continuing need exists for additional and more effective treatment methods. One such alternative envisions methodologies of medical treatment that require or benefit from an ability to initiate, stimulate, and/or enhance an immune response by immunization. These methodologies include those depending upon the creation or stimulation of an immune response against a desired antigenic polypeptide and those that depend upon the initiation or modulation of an innate immune response. Thus one approach in the treatment of cancer is the manipulation of the immune system by use of a therapeutic anticancer vaccine.

### SUMMARY OF THE INVENTION

The instant disclosure relates to methods for treating a cancer patient. Some embodiments of the invention relate to methods of treating a cancer patient with an active immunotherapeutic, wherein the methods include choosing an immunotherapeutic regimen based on a patient's level of pre-existing immunoreactivity to at least one target antigen, wherein the regimen includes administration of at least one immunogen targeting the at least one target antigen, or an epitope thereof; and treating the patient according to the regimen. In some embodiments, the methods described herein include choosing an immunotherapeutic regimen targeting at least one target antigen based on a patient's known level of pre-existing immunoreactivity to the at least one antigen.

In some embodiments, the methods include choosing an immunotherapeutic regimen targeting at least one target antigen based on the presence or absence of immunoreactivity the at least one target antigen in the patient. In some embodiments, the methods include choosing an immunotherapeutic regimen targeting at least one target antigen based on the patient having a substantial or minimal level of immunoreactivity to the at least one target antigen. Some embodiments include a method of treating a cancer patient with an active immunotherapeutic including assessing a patient's level of pre-existing immunoreactivity to at least one target antigen; choosing an immunotherapeutic regimen based on the level of pre-existing immunoreactivity, wherein the regimen includes administration of at least one immunogen targeting the at least one target antigen, or an epitope thereof; and treating the patient according to the regimen.

In some embodiments, the immunogen can promote an effector T cell response to an antigen associated with the cancer. The immunotherapeutic regimen can achieve a clinical benefit. The clinical benefit can include tumor regression or stabilization of disease, and the like, or a combination thereof. The level of pre-existing immunoreactivity can be measured by tetramer or ELISPOT assay, and the like, or a combination thereof.

In some embodiments, the patient can have a cancer that has not progressed beyond secondary lymphatic organs. The patient can have stage IIIC or IV (M1a) lymphatic disease. The cancer can be at least one of, for example, melanoma, kidney, breast, pancreas, prostate, colorectal, ovarian, non-small-cell-lung, glioblastoma, ocular melanoma, hormone sensitive carcinoma of breast, prostate, and ovary, hormone refractory prostate carcinoma, renal cell carcinoma, esophageal, or mesothelioma, and the like. The patient can be HLA-A2 positive.

In some embodiments, the immunotherapeutic regimen for treating a cancer patient can include a prime-boost regimen. In some embodiments, the immunotherapeutic regimen can include more than one therapeutic cycle. The immunogen can be administered direct delivery to the lymphatic system of the patient. The direct delivery to the lymphatic system can include intranodal delivery.

In some embodiments, the prime-boost immunotherapeutic regimen can include administration of an effective amount of a plasmid to induce an immune response followed by administration of an effective amount of at least one peptide corresponding to an epitope expressed by the plasmid. The plasmid can include, for example, pMEL-TYR or pPRA-PSM, and the like. The epitope(s) expressed by the plasmid can include at least one of, for example, a PRAME epitope, or an analogue thereof, or a PSMA epitope, or an analogue thereof, or a Melan A epitope, or an analogue thereof, or a tyrosinase epitope, or an analogue thereof, and the like, or a combination thereof. The epitope(s) expressed by the plasmid can include at least one of, for example, PRAME_{425-433,} or an analogue thereof, or PSMA₂₈₈₋₂₉₇, or an analogue thereof, or Melan A₂₆₋₃₅, or an analogue thereof, or tyrosinase₃₆₉₋₃₇₇, or an analogue thereof, and the like, or a combination thereof. The epitope(s) expressed by the plasmid comprises at least one of Melan A₂₆₋₃₅ A27Nva (ENvaAGIGILTV) (SEQ ID NO:2), or Melan A₂₆₋₃₅ A27L (ELAGIGILTV) (SEQ ID NO:3), or PRAME₄₂₅₋₄₃₃ L426Nva, L433Nle (SNvaLQHLIGNle) (SEQ ID NO:7), or PSMA₂₈₈₋₂₉₇ I297V (GLPSIPVHPV) (SEQ ID NO:9).

In some embodiments, the immunotherapeutic regimen can further include administering an immunopotentiator. In some embodiments, the immunogen can further include an immunopotentiator. The immunopotentiator can include, for example, cytokines, chemokines, co-stimulatory molecules, transcription factors, and signal transduction factors, and the like, or a combination thereof. The immunotherapeutic regimen can further include administering an agent to reduce the immunosuppressive nature of the tumor micro-environment to promote a clinical benefit.

In some embodiments, the target antigen includes Melan A and the patient has a pre-existing immunoreactivity to Melan A. The immunotherapeutic regimen can include administering an immunogen to promote an effector T cell response to Melan A, or one or more epitopes thereof. The patient can have a skin and/or lymphatic disease. The skin and/or lymphatic disease can be, for example, stage IIIC or IV (M1a) lymphatic disease. The immunotherapeutic regimen can include targeting Melan A₂₆₋₃₅ epitope, or an analogue thereof. The cancer can be, for example, melanoma or glioblastoma.

In some embodiments, the target antigen includes at least one of PRAME or PSMA and the patient has no or minimal pre-existing immunoreactivity to the at least one of PRAME or PSMA, and the immunotherapeutic regimen can include administering the immunogen to promote an effector T cell response to the at least one of PRAME or PSMA. PRAME and PSMA can be co-expressed in the patient's tumor tissue. The immunotherapeutic regimen can include targeting at least one of a PRAME₄₂₅₋₄₃₃ epitope or a PSMA₂₈₈₋₂₉₇ epitope. The cancer can be prostate, kidney cancer, or melanoma. The cancer can be prostate cancer and the clinical benefit can include, for example, decreased PSA levels, and the like. The patient may have no or minimal pre-existing immunoreactivity to both PRAME and PSMA; and the immunotherapeutic regimen can further include a subsequent therapy comprising: administering at least one further therapeutic cycle of an active immunotherapeutic targeting at least PRAME or PSMA according to the immunotherapeutic regimen; assaying for expansion of PRAME and/or PSMA T cells in a patient sample; classifying the patient as a responder based on the expansion of antigen specific T cells; and administering at least one further therapeutic cycle to said responder. In some embodiments, the assaying step shows an expansion in PRAME or PSMA T cells and the subsequent therapy can include repeating the therapeutic cycles of the immunotherapeutic regimen. The subsequent therapy can further comprise administering an immunopotentiator. In some embodiments, the assaying step indicates no expansion or a temporary expansion of both PRAME and PSMA T cells and the subsequent therapy can include, for example, discontinuation of the immunotherapeutic regimen.

Some embodiments include a method of treating a cancer patient including: selecting a patient at a stage of disease wherein there is limited spread beyond the lymphatic system and any metastases that have spread beyond the lymphatic system number 10 or fewer and are either 1) not in a vital organ or 2) less than one centimeter in diameter; and applying to the patient an immunotherapeutic regimen including administering directly to the lymphatic system of the patient an immunogen to promote an effector T cell response to an antigen associated with the cancer. The immunotherapeutic regimen can achieve a clinical benefit. The clinical benefit can include tumor regression or stabilization of disease, and the like, or a combination thereof. The patient can have a cancer that has not progressed beyond secondary lymphatic organs. In some embodiments, the patient can have stage IIIC or IV (M1a) lymphatic disease. The cancer can be at least one of, for example, melanoma, kidney, breast, pancreas, prostate, colorectal, ovarian, non-small-cell-lung, glioblastoma, ocular melanoma, hormone sensitive carcinoma of breast, prostate, and ovary, hormone refractory prostate carcinoma, renal cell carcinoma, esophageal, or mesothelioma, and the like. The patient can be HLA-A2 positive. The immunotherapeutic regimen can include a prime-boost regimen. The prime-boost immunotherapeutic regimen can include administration of an effective amount of a plasmid to induce an immune response followed by administration of an effective amount of at least one peptide corresponding to an epitope expressed by the plasmid. The plasmid can include, for example, pMEL-TYR or pPRA-PSM, and the like. The epitope(s) expressed by the plasmid can include at least one of, for example, a PRAME epitope, or an analogue thereof, or a PSMA epitope, or an analogue thereof, or a Melan A epitope, or an analogue thereof, or a tyrosinase epitope, or an analogue thereof, and the like, or a combination thereof. The epitope(s) expressed by the plasmid can include at least one of, for example, PRAME₄₂₅₋₄₃₃, or an analogue thereof, or PSMA₂₈₈₋₂₉₇, or an analogue thereof, or Melan A₂₆-₃₅, or an analogue thereof, or tyrosinase₃₆₉₋₃₇₇, or an analogue thereof, and the like, or a combination thereof. The epitope(s) expressed by the plasmid comprises at least one of Melan A₂₆₋₃₅ A27Nva (ENvaAGIGILTV) (SEQ ID NO:2), or Melan A₂₆₋₃₅ A27L (ELAGIGILTV) (SEQ ID NO:3), or PRAME₄₂₅₋₄₃₃ L426Nva, L433Nle (SNvaLQHLIGNle) (SEQ ID NO:7), or PSMA₂₈₈₋₂₉₇ I297V (GLPSIPVHPV) (SEQ ID NO:9). The immunotherapeutic regimen can further include administering an immunopotentiator. The immunogen can further include an immunopotentiator. The immunopotentiator can include, for example, Toll like Receptor (TLR) ligands, endocytic-Pattern Recognition Receptor (PRR) ligands, cytokines, chemokines, co-stimulatory molecules, transcription factors, and signal transduction factors, and the like, or a combination thereof. The immunotherapeutic regimen can further include administering an agent to reduce the immunosuppressive nature of the tumor micro-environment to promote a clinical benefit. The immunotherapeutic regimen can include more than one therapeutic cycle. The immunogen can be administered direct delivery to the lymphatic system of the patient. The direct delivery to the lymphatic system can include intranodal delivery. In some embodiments, the antigen associated with the cancer includes Melan A and the patient has a pre-existing immunoreactivity to Melan A. The patient can have a skin and/or lymphatic disease. The skin and/or lymphatic disease can be, for example, stage IIIC or IV (M1a) lymphatic disease. The immunotherapeutic regimen can include targeting Melan A₂₆₋₃₅ epitope or an analogue thereof. The cancer can be, for example, melanoma or glioblastoma. In some embodiments, the antigen associated with the cancer includes at least one of PRAME or PSMA and the patient has no or minimal pre-existing immunoreactivity to at least one of PRAME or PSMA. PRAME and PSMA can be co-expressed in the patient's tumor tissue. The immunotherapeutic regimen can include targeting at least one of a PRAME₄₂₅₋₄₃₃ epitope or a PSMA₂₈₈₋₂₉₇ epitope. The cancer can be, for example, prostate, kidney cancer, or melanoma. The cancer is prostate cancer and the clinical benefit can include, for example, decreased PSA levels, and the like. The patient may have no or minimal pre-existing immunoreactivity to both PRAME and PSMA; and the immunotherapeutic regimen can further include a subsequent therapy comprising: administering at least one further therapeutic cycle of an active immunotherapeutic targeting at least PRAME or PSMA according to the immunotherapeutic regimen; assaying for expansion of PRAME and/or PSMA T cells in a patient sample; classifying the patient as a responder based on the expansion of antigen specific T cells; and administering at least one further therapeutic cycle to said responder. In some embodiments, the assaying step shows an expansion in PRAME or PSMA T cells and the subsequent therapy can include repeating the therapeutic cycles of the immunotherapeutic regimen. The subsequent therapy can further comprise administering an immunopotentiator. In some embodiments, the assaying step indicates no expansion or a temporary expansion of both PRAME and PSMA T cells and the subsequent therapy can include, for example, discontinuation of the immunotherapeutic regimen.

Some embodiments include an immunogen for use in the treatment of a cancer in a patient, wherein the patient has a pre-existing immunoreactivity to Melan A, and wherein the immunogen is capable of promoting an effector T cell response to Melan-A. The patient can be HLA-A2 positive. The patient can have a skin and/or lymphatic disease, and wherein the treatment can further include determining the localization of the disease to the lymphatic organs. The skin and/or lymphatic disease can be, for example, stage IIIC or IV (M1a) lymphatic disease. The cancer can be, for example, melanoma or glioblastoma. The treatment can include targeting a Melan A₂₆₋₃₅ epitope.

Some embodiments include an immunogen for use in the treatment of a cancer in a patient, wherein the patient has no or minimal pre-existing immunoreactivity to at least one antigen selected from the group of PRAME and PSMA, and wherein the immunogen is capable of promoting a T cell effector response to the antigen for which there is no or minimal pre-existing immunoreactivity. The patient can be HLA-A2 positive. PRAME and PSMA can be co-expressed in the patient's tumor tissue. The treatment can include targeting a PRAME₄₂₅₋₄₃₃ epitope and/or a PSMA₂₈₈₋₂₉₇ epitope. The cancer can be, for example, prostate cancer and the clinical benefit of the treatment can include, for example, decreased PSA levels, and the like, or a combination thereof. The at least one target antigen can include a PRAME antigen and/or a PSMA antigen and the cancer can be, for example, prostate, kidney cancer, or melanoma. In some embodiments, the patient may have no or minimal pre-existing immunoreactivity to both PRAME and PSMA; and the treatment can further include: administering at least one further therapeutic cycle of an active immunotherapeutic targeting at least PRAME or PSMA; assaying for expansion of PRAME and/or PSMA T cells in a patient sample; classifying the patient as a responder based on the expansion of antigen specific T cells; and administering at least one further therapeutic cycle to the responder. In some embodiments, the assaying step shows an expansion in anti-PRAME or anti-PSMA T cells and the subsequent therapy includes administering subsequent therapeutic cycles of the immunotherapeutic regimen. The subsequent therapy can include, for example, administering an immunopotentiator. In some embodiments, the assaying step indicates no expansion or a temporary expansion of both PRAME and PSMA T cells and the subsequent therapy can include, for example, discontinuation of the immunotherapeutic regimen.

Some embodiments include an immunogen for use as a medicament in the treatment of a cancer in a patient, wherein the patient has a cancer that has not progressed, or has only limited spread, beyond secondary lymphatic organs, and wherein the immunogen is capable of promoting a T cell effector response to an antigen associated with the cancer, wherein the immunogen is for direct intralymphatic administration of the immunogen. The patient can be HLA-A2 positive. The patient can be at a stage of disease wherein there is limited spread beyond the lymphatic system and any metastases that have spread beyond the lymphatic system number 10 or fewer and are either 1) not in a vital organ or 2) less than one centimeter in diameter.

In some embodiments, the pre-existing immunoreactivity or no or minimal immunoreactivity can be measured by, for example, tetramer or ELISPOT assay, and the like, or a combination thereof. The patient can have a cancer that has not progressed, or has only limited spread, beyond secondary lymphatic organs. The patient can have, for example, stage IIIC or IV (M1a) lymphatic disease. The cancer can be at least one of, for example, melanoma, kidney, breast, pancreas, prostate, colorectal, ovarian, non-small-cell-lung, glioblastoma, ocular melanoma, hormone sensitive carcinoma of breast, prostate, and ovary, hormone refractory prostate carcinoma, renal cell carcinoma, esophageal, or mesothelioma, and the like.

In some embodiments, the treatment can achieve a clinical benefit. The clinical benefit can include, for example, tumor regression or stabilization of disease, and the like, or a combination thereof.

In some embodiments, the treatment can include a prime-boost regimen. The treatment can include more than one therapeutic cycle. The prime-boost immunotherapeutic regimen can include administration of an effective amount of a plasmid to induce an immune response followed by administration of an effective amount of at least one peptide corresponding to an epitope expressed by the plasmid. The epitope(s) expressed by the plasmid can be at least one of, for example, a PRAME epitope or an analogue thereof, or a PSMA epitope or an analogue thereof, or a Melan A epitope or an analogue thereof, or a tyrosinase epitope or an analogue thereof, and the like, or a combination thereof. The PRAME epitope can be, for example, PRAME₄₂₅₋₄₃₃ or an analogue thereof. The PSMA epitope can be, for example, PSMA₂₈₈₋₂₉₇ or an analogue thereof. The Melan A epitope can be, for example, Melan_{A26-35}, or an analogue thereof. The tyrosinase epitope can be, for example, tyrosinase₃₆₉₋₃₇₇ or an analogue thereof. The plasmid can include, for example, pMEL-TYR or pPRA-PSM. The at least one peptide can include, for example, Melan A₂₆₋₃₅ A27Nva (ENvaAGIGILTV) (SEQ ID NO:2) or Melan A₂₆₋₃₅ A27L (ELAGIGILTV) (SEQ ID NO:3), PRAME₄₂₅₋₄₃₃ L426Nva, L433Nle (SNvaLQHLIGNle) (SEQ ID NO:7), or PSMA₂₈₈₋₂₉₇ peptide analogue PSMA₂₈₈₋₂₉₇ I297V (GLPSIPVHPV) (SEQ ID NO:9), and the like, or a combination thereof.

In some embodiments, the immunogen can be for direct delivery to the lymphatic system of the patient. The direct delivery to the lymphatic system can include, for example, intranodal delivery.

In some embodiments, the treatment can further include administering an immunopotentiator. The immunogen can further include an immunopotentiator. The immunopotentiator can include, for example, cytokines, chemokines, co-stimulatory molecules, transcription factors, signal transduction elements; agents that are involved in antigen processing and presentation, TAP 1 and TAP 2 proteins, immune or standard proteasome, beta-2-microglobulin, and MHC class I or II molecules; agents that are involved in regulating the apoptotic pathway, agents that are involved in gene control or silencing, such as DNA methylating enzymes, chromatin controlling molecules, RNA regulating molecules; Toll-like receptors (TLRs), peptidoglycans, LPS or analogues therefrom, imiquimodes, unmethylated CpG oligodeoxynuclotides (CpG ODNs); dsRNAs, bacterial dsDNA (which contains CpG motifs), and synthetic dsRNA (polyI:C) on APC and innate immune cells that bind to TLR9 and TLR3, respectively; small organic natural or synthetic molecules that bind to TLRs, synthetic anti-viral imidazoquinolines, imiquimod and resiquimod; immunopotentiating adjuvants that activate pAPC or T cells, endocytic-Pattern Recognition Receptor (PRR) ligands, quillaja saponins, and tucaresol, and the like, or a combination thereof. The treatment can further include administering an agent to reduce the immunosuppressive nature of the tumor microenvironment to promote a clinical benefit.

### BRIEF DESCRIPTION OF THE DRAWINGS

FIG. 1 depicts a plasmid and peptide schedule of administration used in exemplary embodiments of the disclosure.

FIG. 2 depicts a plasmid design for the pMel-pTyr plasmid and the Melan A and tyrosinase peptides for administration.

FIG. 3 shows a 59% tumor shrinkage lasting 48+ weeks.

FIG. 4 shows a 60% tumor shrinkage lasting 36+ weeks.

FIG. 5 shows a 30% tumor shrinkage lasting 36+ weeks.

FIGS. 6A-6B shows Melan A/MART-1 T cells at baseline with disease stage and clinical outcome. FIG. 6A shows subjects with tumor response (lymphatic disease) or no tumor response at the disease stage (stage IV(M1b, c) or stage IIIC and IV(M1a)). FIG. 6B shows the clinical outcome (PD, PR or SD) of subjects in FIG. 6A. (†SD for 2 cycles. ‡Subjects with ≧1 year duration of treatment.) Closed circles represent subjects with no tumor response. Open circles represent subjects with tumor response (lymphatic disease).

FIG. 7 shows the expansion of epitope-specific T cells in peripheral blood as measured by MHC multimer staining (tetramer assay).

FIG. 8 depicts a plasmid design for the pPRA-PSM plasmid and the PRAME and PSMA peptides for administration.

FIG. 9 shows a tumor shrinkage lasting 36 weeks.

FIG. 10 shows association of clinical benefit with the expansion of epitope-specific T cells as measured by MHC multimer staining.

FIG. 11 shows association of immune response with clinical outcome. PC: prostate carcinomas, RCC: renal carcinomas, Mel: melanomas, ESO: Esophageal, * Epitope specific T cells at baseline detected.

FIG. 12 depicts a comparison of the evaluations of tumor growth by the trial investigators (open bars) and that of an independent radiologist (black bars) showing qualitative agreement on a trend of tumor reduction or stabilization in patients with disease predominantly localized to lymph nodes and tumor progression in patients with visceral metastatic disease.

### DETAILED DESCRIPTION

### Definitions

Unless otherwise clear from the context of the use of a term herein, the following listed terms shall generally have the indicated meanings for purposes of this description.

As used herein, “treatment” or “treating” refers to the act of treating a patient medically, such as by administration or application of remedies to a patient.

As used herein, “immunotherapeutic regimen, treatment, or protocol” refers to a plan for a medical treatment or a desired course of treatment. The regimens, treatments, or protocols for use in the methods described herein are therapeutic regimens for use in clinical or medical settings. The regimen or protocol can specify any part of or all of the schedule of administration, the route of administration, and the specific reagents to be administered.

As used herein, “active immunotherapy” or “active immunotherapeutic” refers to procedures or reagents to stimulate the body's own immune system to fight a disease. Inclusive herein are active immunotherapeutic agents, compositions or products. The active immunotherapeutic refers to an immunogen or an immunogenic agent or product or composition that can be used as an immunotherapeutic or therapeutic, for example by providing or aiding in treatment of disease. In some embodiments, the active immunotherapeutic agent, product or composition can provide or aid in curing of a disease. In some embodiments, the active immunotherapeutic agent, product or composition provide or aid in amelioration of a disease.

As used herein, “immunization” refers to a process to induce or amplify an immune system response to an antigen. Alternatively, it refers to a process to reduce/decrease progression of cancer, or to prevent/block metastases of such a disease by the immune system. In the first definition it can connote a protective and/or therapeutic immune response, particularly proinflammatory or active immunity, though it can also include a regulatory response.

As used herein, “immunoreactivity” refers to a biochemical interaction between an immune receptor and an antigen, for purposes of the instant invention particularly a T cell receptor (TCR) and a MHC/antigen complex, respectively. In some embodiments, immunoreactivity can be part of, or the result of, an immune response.

As used herein, “targeting a target antigen” refers to promoting an immune response that recognizes the target antigen or at least one particular epitope thereof. Similarly, “targeting a tumor or cell” refers to promoting an immune response that recognizes an antigen or at least one particular epitope thereof expressed by the targeted tumor or cell. In some embodiments, the immune response is an effector T cell response. In some embodiments, the effector T cell response includes a cytotoxic T cell (CTL) response. In some embodiments, the therapeutic effect of the active immunotherapeutic disclosed herein is mediated by effector T cells.

A “target cell,” as used herein, refers to a cell associated with a cancerous condition that can be acted upon by the components of the immune system, such as, for example, a neoplastic cell or a cell associated with tumor neovasculature. In some embodiments, a target cell is a cell to be targeted by the vaccines and methods disclosed herein. A target cell according to this definition includes, but is not limited to, a neoplastic cell. In further embodiments, a target cell is a cell that expresses the target antigen or presents the target epitope.

As used herein, a “target-associated antigen (TAA)” refers to a protein or polypeptide present in a target cell.

As used herein, a “tumor-associated antigen (TuAA)” refers to a TAA, wherein the target cell is a tumor-associated cell. In various embodiments, a TuAA is an antigen associated with, a neoplastic cell, or non-cancerous cells of the tumor such as tumor neovasculature or other stromal cells within the tumor microenvironment.

The term “epitope” refers to a site on an antigen recognized by an antibody or an antigen receptor. A T cell epitope is generally a short peptide derived from a protein antigen (though non-protein antigens are also known). T cell epitopes bind to MHC molecules and are recognized by a particular T cell. In some embodiments, an epitope can include, but is not limited to, peptides presented on the surface of cells, the peptides being non-covalently bound to the binding cleft of class I MHC, such that they can interact with T cell receptors (TCR).

As used herein, “immunopotentiators” or “immunopotentiating adjuvant” refers to any molecule that enhances, increases, promotes, or up-modulates the activity of the immune system or the cells thereof, and particularly their effector functions, through an interaction other than with an antigen receptor. An “immunopotentiating adjuvant” refers to an adjuvant that activates professional antigen presenting cells (pAPCs) or T cells including, for example: TLR ligands, endocytic-Pattern Recognition Receptor (PRR) ligands, quillaja saponins, tucaresol, cytokines, and the like. Some preferred adjuvants are disclosed in Marciani, D., J. Drug Discovery Today 8:934-943, 2003, which is incorporated herein by reference in its entirety.

As used herein, “therapeutic cycle” or “cycle” refers to one or more priming/inducing/entraining doses followed by one or more boosting/amplifying doses. This sequence can be repeated as long as necessary to maintain a strong immune response in vivo. Thus, in some embodiments, a therapeutic cycle includes two or more priming/inducing/entraining doses followed by two or more boosting/amplifying doses. In some embodiments, a therapeutic cycle or cycle includes one priming/inducing/entraining dose followed by one or more boosting/amplifying doses. In other embodiments, one therapeutic cycle includes one or more priming/inducing/entraining dose followed by one boosting/amplifying doses. The immunotherapeutic regimen, protocol or treatment disclosed herein can generally involve one or multiple cycles (such as 2 or more, 4 or more, 6 or more, 8 or more, 10 or more or so on).

As used herein “target lesion” refers to a tumor in a patient of sufficient size that it can be evaluated for change in size, especially shrinkage, in response to a cancer therapy. Tumor size can be determined, for example, by tumor imaging or direct measurement. Tumor imaging can be accomplished by CT scan, MRI, and/or PET scan, and the like. Skin lesions can be directly measured. In some embodiments, target lesions measure greater than 1 cm in their largest dimension at start of treatment or evaluation.

As used herein “non-target lesion,” used in contradistinction to “target lesions,” are tumors that measure less than 1 cm in their largest dimension at start of treatment or evaluation.

As used herein “lymphatically confined,” “not progressed beyond the lymphatic system” and similar constructions are to be understood in the context of the typical progression of malignant disease in which there is first local spread proximate to the site of origin either within the same tissue or organ or into adjacent tissues or organs followed by spread through the secondary lymphatic organs, particularly to lymph nodes locally or systemically and later to distant visceral sites. In some instances there are also skin or subcutaneous metastases prior to the appearance of visceral metastases. The terms “lymphatically confined” and/or “not progressed beyond the lymphatic system” as used herein can be used to refer to the actual limit of disease progression in a patient or the state of the disease following surgical removal and/or other interventions. Thus, these terms can refer to any point in disease progression prior to spread to the viscera. In some embodiments, these terms can be used to refer to patients at more specifically defined stages of disease as described herein. In some embodiments, a patient is characterized as having lymphatically confined disease due to the removal or ablation of visceral lesions following surgery or other treatment. In some embodiments, a patient is characterized as having disease that is “primarily lymphatically confined” or as having only “limited progression beyond the lymphatic system” (and the like) based on the presence of visceral metastases that are limited in size and/or number as compared to a pre-determined size and/or number.

The instant disclosure relates to a significant advancement in the utilization of active immunotherapies against carcinomas and the discovery and use of mechanistic-based, companion biomarkers, such as pre-existing immunoreactivity, to more advantageously utilize immunogenic products that induce, promote the growth of, activate, and/or otherwise stimulate effector T cells targeting tumor antigens in the treatment of cancer. Exemplary advancements provided by the methods and immunogens disclosed herein include, but are not limited to, identification of patient subset(s) in which immunotherapy is predictably effective; achievement of durable objective responses with a cancer vaccine at a readily observable rate; first observation of selective potency of lymphatically administered immunotherapeutics against lymphatic metastatic disease; prognostic value of pre-existing immune status to target antigen(s) in predicting effectiveness of immunotherapy (at least against lymphatic metastatic disease); prolonged progression free survival in metastatic disease within patient subsets.

In some embodiments, the disclosure relates to methods of identifying or selecting patients for an immunotherapeutic regimen that involves the administration of an agent that generates an effector T cell response against a target antigen. For example, the effector T cell response can be a CTL response. In some embodiments, the disclosure relates to a method of treating a patient having a carcinoma wherein pre-existing immunoreactivity to at least one or more target antigens is assessed in a patient prior to selection for an immunotherapeutic regimen. For example, in some embodiments, a patient is selected for treatment with a particular immunotherapeutic or immunotherapeutic regimen on the basis of having or not having a pre-existing immunoreactivity to a particular TuAA or epitope thereof. In the methods disclosed herein, selecting or identifying a patient can involve assessing the level (e.g., the presence or absence) of a pre-existing immunoreactivity to at least one target antigen—i.e. the presence or absence of T cells that recognize a target antigen, or at least one particular epitope thereof—prior to initiation of the therapeutic regimen. The presence or absence of a pre-existing immunoreactivity against a target antigen can be measured, for example, by MHC multimer staining and/or ELISPOT assay, and/or the like. In some embodiments, selecting or identifying includes selecting or identifying a patient having no, or minimal pre-existing immunoreactivity to the target antigen. In some embodiments, selecting or identifying includes selecting or identifying a patient having a pre-existing immunoreactivity to the target antigen. The immunoreactivity or the immunity of the patient against a target antigen is measured, and compared to a predetermined value. If the immunoreactivity is above or equal to the predetermined value, the patient is categorized as having a pre-existing immunoreactivity against the target antigen. If the immunoreactivity is below the predetermined value, the patient is categorized as having no or minimal pre-existing immunoreactivity to the target antigen. The predetermined value can be chosen based on considerations including, for example, the technique employed to measure the value (for example, MHC multimer staining, and/or ELISPOT assay, and/or the like), the “normal value of immunoreactivity” against the target antigen, and the like, or a combination thereof. The “normal value of the immunoreactivity” can be, for example, that of the same patient before the patient acquires the disease at issue, or the average value of healthy people (for example, people without the disease at issue) of a specific region, age group, gender, and/or the like. In some embodiments, a patient with pre-existing immunoreactivity against a target antigen has a number of antigen specific T cells at least 3 times greater than the lower limit of detection of the assay. Conversely, a patient lacking pre-existing immunoreactivity against a target antigen has a number of antigen specific T cells less than 3 times the lower limit of detection of the assay. In an exemplary embodiment, the lower limit of detection of the MHC multimer staining (tetramer assay) is 0.03 percent of tetramer positive CD8+ T cells per total number of CD8+ T cells.

Embodiments disclosed herein relate to methods of treatment wherein a patient whose disease has not spread beyond the lymphatic system is selected for treatment with a regimen that includes direct intralymphatic administration of an immunotherapeutic reagent. In an exemplary embodiment, administration is to a non-diseased lymph node. Thus, in some embodiments, the methods disclosed herein include selecting or identifying a patient having a metastatic disease that is lymphatically confined (either because the disease has not progressed beyond the lymphatic system or is confined following surgery or other treatment) or that is beyond the lymphatic system only to a minimal degree (e.g., having metastatic lesions beyond the lymphatic system but not in a vital organ, for example, but not limited to, soft tissue lesions, wherein such lesions are ten or fewer in number; or metastatic lesions not necessarily excluding those in vital organs that are less than 1 cm in diameter). In some embodiments, the methods include selecting or identifying a patient having a metastatic disease that has not progressed to the presence of visceral metastases. Generally, a patient with an invasive disease at the site of origin is not excluded from these groups though in some embodiments, they are. In some embodiments, selecting or identifying according to the methods disclosed herein includes selecting or identifying a melanoma patient having only skin lesions.

Thus, in some embodiments, the methods disclosed herein include selecting or identifying a patient having metastatic disease/lesions outside of or beyond the lymphatic system. In some embodiments, such metastatic lesions that have spread beyond the lymphatic system have spread to for example, soft tissue but not to a vital organ, and the lesions are ten or fewer than ten in number. In some embodiments, there are 10, 9, 8, 7, 6, 5, 4, 3, 2, or 1 lesions. In some embodiments, the metastatic lesions beyond the lymphatic system are less than one centimeter in diameter. In some embodiments the lesions less that one centimeter in diameter can be in a vital organ.

In some embodiments, the methods disclosed herein include selecting or identifying a patient having no metastases beyond the organ or tissue of disease origin, or in which there has been spread only to the adjacent organs or tissues. In some embodiments, a patient whose disease has spread beyond the lymphatic system to a non-vital organ and whose metastatic lesions are ten or fewer lesions, is selected for treatment with an immunotherapeutic pursuant to the methods disclosed herein.

### Selection of Patients

As discussed herein, patients, in general, can be selected based on the status of HLA type, the level of pre-existing immunoreactivity to a target antigen, and/or whether the disease is lymphatically confined or has progressed beyond the lymphatic system. Thus, in some embodiments, patient selection can include being positive for a particular HLA type where the immunotherapeutic regimen generates a response restricted by that HLA type, for example, but not limited to, HLA-A2, or HLA-A*0201.

In some embodiments, patients are selected according to whether a pre-existing immunoreactivity to a target antigen is substantial, present, minimal, or absent as compared to baseline or a predetermined value as discussed herein. The presence or absence of a pre-existing immunoreactivity against a target antigen can be measured, for example, by MHC multimer staining, and/or ELISPOT assay, and/or the like. For example, in some embodiments, the methods disclosed herein involve selecting a patient with a pre-existing immunoreactivity to a target antigen. Thus, patients whose immunoreactivity against a target antigen is at, or above, or substantially above a predetermined value are selected for administration of an active immunotherapeutic composition targeting the target antigen or one or more epitopes thereof. For example, in some embodiments, patients having a baseline pre-existing immunoreactivity to a target antigen that is greater than or substantially above a predetermined value, for example, greater than about 0.1% of CD8⁺ T cells as determined by MHC multimer staining analysis are selected. Thus, in some embodiments, patients having a baseline immunoreactivity to the target antigen that is greater than a predetermined value, for example, greater than about 0.10%, about 0.11%, about 0.12%, about 0.13%, about 0.14%, about 0.15%, about 0.16%, about 0.17%, about 0.18%, about 0.19%, and about 0.20% (% T cells stained out of all CD8+ T cells) as determined by MHC multimer analysis are selected for administration of an active immunotherapeutic composition targeting the target antigen, or one or more epitopes thereof.

In some embodiments, the methods involve selecting a patient with no, or minimal, pre-existing immunoreactivity to the target antigen. Thus, patients whose immunoreactivity against a target antigen falls at or below a predetermined value are selected for administration of an active immunotherapeutic composition targeting the target antigen or one or more epitopes thereof. For example, in some embodiments, patients having a baseline pre-existing immunoreactivity to a target antigen of less than a predetermined value, for example, less than about 0.1% of CD8⁺ T cells as determined by MHC multimer analysis are characterized as having no, or minimal, preexisting immunity and are selected for administration of an active immunotherapeutic composition targeting the target antigen, or one or more epitopes thereof. For example, patients having a baseline immunoreactivity to the target antigen of less than a predetermined value, for example, less than about 0.10%, about 0.09%, about 0.08%, about 0.07%, about 0.06%, about 0.05%, about 0.04%, about 0.03%, about 0.02%, or about 0.01%, or not distinguishable from background levels as determined by MHC multimer analysis are selected for treatment.

As will be apparent to those of skill in the art, the predetermined values are dependent upon the technique used to measure immunoreactivity and the response being sought. Based on the disclosure herein, these values will be apparent to those of skill in the art for a particular type of assay or measurement, as will additional or alternate classifications/stratifications useful for the methods described herein.

In some embodiments, the selecting or identifying further includes selecting or identifying a patient having a metastatic disease that has not progressed beyond a predetermined stage. The stage of a cancer is a descriptor of how much the cancer has spread. As will be understood by those of skill in the art, although the specific terminology differs for the staging of various cancers, the general underlying principles are the same.

It is meaningful to speak of cancers that are localized to the site or organ or tissue of origin, have invaded surrounding normal tissue, have invaded the lymphatic system, have invaded the veins and capillaries, have or have not metastasized to distant/visceral organs, for any type of carcinoma. For melanoma, for example, the stage often takes into account the size and/or thickness of a tumor, how deep it has penetrated the dermal layers or subcutaneous tissue, whether it has invaded adjacent organs, how many lymph nodes it has metastasized to (if any), and whether it has spread to distant organs. The letters TNM (Tumor-Node-Metastasis) are used to further define the staging of melanoma for example, wherein the letter T represents the thickness of the tumor; the letter N represents to the number of lymph nodes involved; and the letter M represents metastasis to distant organs. Each of these letters can include a numerical value (e.g., 0, 1 etc.) to indicate the thickness, number of lymph nodes or extent of metastasis respectively. For example, in evaluating metastasis the numerical value used is, ‘0’ if no metastasis has occurred or ‘1’ if metastases are present. (See, e.g., Restas, S. and Mastrangelo, M J, Seminars in Oncology 2007 34(6):491-497, which is hereby incorporated by reference in its entirety.)

The stages of melanoma, for example, are stage 0, I, II, III or IV at least partially dependent on localization of the diseased tissue (for example, the tumor tissue) and in some stages can include the lymphatic organs. As used herein, the lymphatic organs include, for example, the spleen, lymph nodes, lymph vessels, and the like. Stage 0, IA, IB, melanoma take into account the tumor size or thickness; do not involve metastasis, and are highly curable. Stage IIA, IIB and IIC melanoma shows increase tumor size or thickness than observed at stage I, do not involve the lymph nodes, and is curable. Stages IIIA, IIIB and IIIC melanoma refer to an advanced stage which takes into account the tumor size or thickness, whether one or more lymph nodes are involved and that the cancer has begun to metastasize. Stage IV melanoma is another advanced stage which, in addition to the characteristics taken into account for Stage III melanoma, is associated with metastasis, represented by ‘M1’—distant metastasis. For example, for melanoma, stage IV M1a indicates that the melanoma has not progressed beyond the lymph nodes, (i.e.lymphatic disease), is limited to distant skin and subcutaneous tissues or lymph nodes whereas disease which has progressed beyond the lymph node to the M1b or M1c stage is representative of visceral disease i.e. metastasis has occurred to the lung, or any other visceral sites, or distant metastasis with elevated levels of serum lactic dehydrogenase (LDH) at any site.

The stages of renal cell carcinoma or kidney cancer are stage I, II, III or IV. Stages I and II, take into account the tumor size or thickness, and do not involve the metastasis. Stage III renal cell carcinoma takes into account the tumor size or thickness, whether one or more nearby lymph nodes are involved, and whether the cancer has invaded the blood vessels of the kidney or the surrounding fatty tissue. Stage IV renal cell carcinoma is an advanced stage which, in addition to the characteristics taken into account for Stage III of the disease, is associated with metastasis to the adrenal gland, lung, liver, bone, or brain.

The stages of prostate cancer are stage I, II, III or IV. Stage I prostate cancer takes into account whether the degree of spread of the tumor is to one-half, or less, of one lobe of the prostate and that the level of PSA (prostate specific antigen) is less than 10 nanograms per milliliter. In Stage IIA prostate cancer, the degree of spread of the tumor is similar to that of Stage I or to more than half of one lobe of the prostate, and the level of PSA is at least 10 nanograms per milliliter but less than 20 nanograms per milliliter. In Stage IIB prostate cancer, the degree of spread of the tumor is to both lobes of the prostate, and the level of PSA is 20 nanograms per milliliter or higher. In Stage III prostate cancer, the tumor has spread beyond the prostate to the seminal vesicles and the level of PSA varies. In Stage IV prostate cancer, advanced stage disease, the tumor has spread beyond the seminal vesicles to nearby lymph nodes, tissues or organs such as the bladder, pelvis or rectum, or metastasized to distant lymph nodes, organs or tissues including the bone; PSA levels are varied.

In some embodiments, the active immunotherapeutic product disclosed herein can be used to treat disease, such as melanoma or glioblastoma, localized to the lymph nodes. In some embodiments, the active immunotherapeutic disclosed herein is used to treat a disease stage of cancer (such as, for example, glioblastoma, melanoma, prostate, renal cell carcinoma) wherein there is limited spread of disease beyond the lymphatic system, or the disease is otherwise (predominantly) confined to the lymphatic system following surgery or other treatment, and any metastases (lesions) that have spread beyond the lymphatic system are ten or fewer in number, and are either not in a vital organ or less than one centimeter in diameter if involving a vital organ. Thus, in some embodiments, metastases have spread or progressed beyond the lymphatic system but not to a vital organ (such examples of vital organs include lung, liver, adrenal gland) and such metastatic lesions are ten or fewer than ten in number. In some embodiments, the metastatic lesions have spread beyond the lymphatic system, including in some embodiments, spread to a vital organ, and such metastatic lesions are less than one centimeter in diameter.

In some embodiments, the active immunotherapeutic disclosed herein can be used to treat clinically overt (macroscopic) disease, (for example, disease such as advanced melanoma), measured utilizing conventional imaging techniques such as computed tomography (CT) scanning, magnetic resonance imaging (MRI), positron emission tomography (PET), bone scan, or X-ray. In some embodiments, the active immunotherapeutic product can be used to treat microscopic (i.e. disease detectable by biopsy but not by imaging techniques) disease, minimal residual disease and prevent or delay disease relapse. In some embodiments, the immunotherapeutic product disclosed herein is utilized to prevent development of clinically overt metastatic lesions.

Exemplary immunotherapeutic regimens disclosed herein which include direct administration to the secondary lymphatic system were seen to be particularly effective against cancers that had not yet progressed beyond the lymphatic system, or had done so to only a limited extent. While intralymphatic immunization is known to be advantageous in regard to sensitivity to immunogen, for example, an advantage relating to the anatomical location of the target disease was neither predictable nor expected. Thus in some embodiments, a patient with lymphatically confined disease or primarily lymphatically confined disease is selected for treatment under an immunotherapeutic regimen including intralymphatic administration of an immunogen targeting a tumor-associated antigen. In some embodiments, direct administration to a lymph node is utilized. In some embodiments, the cancer has not yet spread into the lymphatic system. In some embodiments, the treatment inhibits or prevents spread of the disease, for example spread of the disease beyond the lymphatic system.

### Measuring Immunoreactivity

In embodiments of the methods disclosed herein, methods to rapidly and reliably assess a patient's immune response to a component or multiple components of an active immunotherapeutic composition are used. Some embodiments include defining or classifying a patient as a positive immune responder versus a non-responder. For example, the patient's immune response to at least one immunizing antigen (or target antigen) can be measured by MHC multimer staining, and/or ELISPOT assay, and/or the like, and predetermined criteria for classifying the patient as a positive immune responder can be a two-fold greater MHC multimer staining and/or three-fold greater ELISPOT reaction relative to a pretreatment baseline and significantly different from assay background. Pre-existing immunoreactivity can be assessed versus assay background. Such methodologies are well known in the art. Enzyme-linked immunospot (ELISPOT) is a sensitive technique for the detection of biomarkers released by cells or detection of individual cells that secrete a biomarker of interest, such as for example, cells secreting antibodies, cytokines, chemokines, or granzymes. The technique is well established and correlates closely with the enzyme-linked immunosorbent assay (ELISA) technique. (Vaquerano et al., Biotechniques. 1998 November; 25(5):830-4, 836, which is hereby incorporated by reference in its entirety.)

As used herein, a MHC multimer staining (e.g., tetramer assay) refers to the method to detect the presence of antigen-specific T cells wherein MHC-peptide complexes that have been multimerized and attached to a reporter molecule, typically a fluorescent dye, are contacted with T cells, binding to those expressing a T cell receptor (TCR) that recognizes the MHC-peptide complex and allowing their detection through the reporter molecule. Merely by way of example, the multimer can include a dimer, a pentamer, a tetramer, or the like. Such reagents are commercially available. Also as used herein, an ELISPOT assay (or IFN-gamma enzymatic linked immune spot assay) refers to the method used to assess the CD8⁺ CTL response by measuring IFN-gamma production by specific effector cells in an ELISPOT assay. In this assay, cells are immobilized on the plastic surface of a microtiter well and effector cells are added. The binding of cells by antigen-specific effector cells triggers the production of cytokines including IFN-gamma by the effector cells. The cells are then stained to detect the presence of intracellular IFN-gamma and the number of positively staining foci (spots) counted under a microscope.

In some embodiments, response can be assessed based on the phenotype of antigen-specific T cells using, for example, flow cytometry to detect surface markers associated with different phenotypes. For example, an increase in the proportion of antigen-specific T cells that have an effector phenotype or an effector/memory phenotype can be taken as a positive response even in the absence of an increase in the total number of antigen specific T cells. Such practice is well known in the art and appropriate reagents are commercially available.

Other methods for determining or assessing an immune response to an active immunotherapeutic composition as disclosed herein include, for example, but not limited to, intracellular cytokine staining (ICS assay); DTH response—preferably an antigen-specific DTH, cytokine assays, cell proliferation assays, chromium release assays, and/or the like. Many technologies to carry out such assays are known in the art. Such assays can be specific for a target epitope, not just the antigen, and thus can be referred to as epitope determinations. Reagents that detect presentation of particular T cell epitopes from target antigens can also be used. These include, for example, T cell lines and hybridomas, and more preferably, antibodies specific for the peptide-MHC complex and TCR multimers (see, for example, Li et al., Nature Biotech. 23:349-354, 2005, which is incorporated herein by reference in its entirety). Appropriate reagents are commercially available.

In some embodiments, a patient sample, such as blood, or other bodily fluids or secretions, biopsied tumor tissue, or portions thereof, such as lymphocytes or cytokines, is assayed for an immune response. In some embodiments, the immune response is measured using visual observations of the body, such as a skin test for DTH.

In some embodiments, only a desired immune response(s) is assayed for. In some embodiments, only an undesired immune response(s) is assayed for. In still some embodiments, both desired and undesired immune responses are assayed for. In some embodiments, the response includes, in a non-limiting manner, tumor shrinkage or reduction or regression, tumor clearance, inhibition of tumor progression, amelioration of the cancer, minimal residual disease, clinical response (as measured by Response Evaluation Criteria in Solid Tumor (RECIST) criteria), and the like. In some embodiments, the patient is classified as a responder based on expansion of antigen specific T cells after immunization, for example after at least two cycles of an immunotherapeutic regimen and/or achievement of a clinical benefit. In particular embodiments the patient, for example a prostate cancer patient, is classified as a responder based on tumor regression mirrored by PSA decline.

### Pre-Existing Immunoreactivity to Melan A

In evaluating immunity and clinical outcomes of active immunotherapy in patients with advanced melanoma, tumor responses (e.g., tumor shrinkage) were observed in subjects having a pre-existing immunoreactivity to Melan A as measured by MHC multimer staining for the targeted Melan A epitope. That is, clinical outcomes and/or tumor responses were predicted by a pre-existing Melan A/MART-1 T cell response prior to or at the time of initiation of the therapeutic regimen. As can be determined by one of ordinary skill in the art, pre-existing immunoreactivity can be measured by other means disclosed herein and known in the art.

Thus, some embodiments relate to methods of treating a patient having a carcinoma, such as, melanoma, (or a cancer, such as, glioblastoma), the method including selecting a patient with a pre-existing immunoreactivity to at least one target antigen or one or more epitopes thereof, wherein the target antigen is Melan A, and administering to the patient an active immunotherapeutic composition targeting Melan A or one or more epitopes thereof. In some embodiments, the immunotherapeutic composition also targets at least one additional antigen associated with the tumor, or one or more epitopes thereof. In some embodiments, the additional tumor associated antigen is tyrosinase.

In some embodiments, patients having disease confined to the lymphatic system (for example, M1a stage metastatic disease—the stages of disease are described elsewhere herein) show clinical benefit. In some embodiments, the methods disclosed herein include selecting a patient having a carcinoma that has not progressed beyond the lymphatic system or lymph nodes. In some embodiments, a patient whose disease has spread beyond the lymphatic system but not to vital organs and whose lesions are ten or fewer in number, shows clinical benefit. In some embodiments, there are lesions in vital organs but they are less than one centimeter in diameter.

As demonstrated herein, a pre-existing immunoreactivity to Melan A, or a particular epitope thereof, has been associated with a positive clinical outcome upon treatment with an immunotherapeutic targeting Melan A. Thus, in some embodiments, a patient is selected to receive an active immunotherapeutic composition targeting Melan A based on a patient having a pre-existing immunoreactivity to Melan A. Thus, in some embodiments, a patient with pre-existing immunoreactivity to a target antigen is selected for treatment with an immunotherapeutic regimen for which a positive clinical outcome is associated with such pre-existing immunoreactivity. In some embodiments, the target includes Melan A and the patient has no or minimal pre-existing immunoreactivity to Melan A and the immunotherapeutic regimen includes co-administering an immunogen and an immunopotentiator to promote an immune response (e.g., an effector T cell response) to Melan A.

In some embodiments, the carcinoma is a Melan A positive cancer, such as, for example, melanoma or glioblastoma, and the like, and the at least one target antigen is Melan A. In some embodiments, the immunotherapeutic agent or composition can also target additional antigens associated with the tumor, for example, but not limited to, tyrosinase, or one or more epitopes thereof. The immunotherapeutic regimen can involve a prime-boost protocol. In some embodiments, a particular epitope is targeted. In some embodiments, the targeted epitope includes Melan A₂₆₋₃₅. In particular embodiments, the components of the immunotherapeutic regimen are delivered separately and intranodally.

In some embodiments, patients having a baseline pre-existing immunoreactivity to Melan-A target antigen of greater than a predetermined value, for example, about 0.1% of CD8⁺ T cells as determined by MHC multimer staining analysis are selected. In some embodiments, and merely by way of example, patients with a baseline immunoreactivity to a target antigen of greater than a predetermined value, for example, greater than about 0.10%, about 0.11%, about 0.12%, about 0.13%, about 0.14%, about 0.15%, about 0.16%, about 0.17%, about 0.18%, about 0.19%, about 0.20% (% T cells stained out of all CD8+ T cells) as determined by MHC multimer analysis are characterized as having a pre-existing immunoreactivity to Melan-A. Patients with pre-existing immunoreactivity to the target antigen of less than a predetermined value, for example, less than about 0.10%, or about 0.09%, or about 0.08%, or about 0.07%, or about 0.06%, or about 0.05%, or about 0.04%, or about 0.03%, or about 0.02%, or about 0.01% of CD8⁺ T cells are characterized as having no or minimal immunoreactivity against Melan-A. A person of ordinary skill in the art, reading the instant application, would understand that the predetermined value against which the baseline immunoreactivity is compared can be chosen based on the specific assay that is used to evaluate immunoreactivity.

### Pre-Existing Immunoreactivity to PRAME and PSMA

In evaluating immunity and clinical outcomes of active immunotherapy in patients with PRAME and PSMA positive solid tumors, positive clinical observations (including, but not limited to, stable disease, enablement of apparently curative surgery, reduction in PSA titers or velocity) can be associated with the lack of a pre-existing immunoreactivity to the targeted antigens, or epitopes thereof, and also with expansion or persistence of T cells in the blood during the first two cycles of treatment. A proportion of patients showing clinical benefit showed expansion of antigen-specific T cells in the blood early on after initiation of the therapeutic regimen. In some embodiments, a positive clinical outcome can be predicted by a de novo induction of a T cell response with effector cells. Thus, some embodiments involve a theranostic approach (see for example U.S. patent application Ser. No. 11/155,928 (Publication No. US 2005-0287068 A1), which is hereby incorporated by reference in its entirety). Thus, in some embodiments, failure to expand or maintain a response to either antigen is used as a basis for either discontinuing treatment with the immunotherapeutic agent/composition or modifying the treatment to a more strongly immunogenic protocol, for example, to include administration of an adjuvant, including an immunopotentiating adjuvant.

In some embodiments, patients having disease such as, for example, prostate cancer or renal cell carcinoma, confined to the lymphatic system show clinical benefit. In some embodiments, the methods disclosed herein include selecting a patient having a carcinoma that has not progressed beyond, or is otherwise confined to, the lymphatic system or lymph nodes. In some embodiments, a patient whose disease has spread beyond the lymphatic system but not to vital organs and involves ten or fewer than ten lesions, shows clinical benefit. In some embodiments, there are lesions in vital organs but they are less than one centimeter in diameter.

Some embodiments relate to methods of treating a patient having a carcinoma, the methods including selecting a patient with no, or minimal, pre-existing immunoreactivity to PRAME and/or PSMA or a particular epitope of PRAME and/or PSMA, for example, as measured by MHC multimer staining, and/or ELISPOT assay, and/or the like, and administering to the patient an immunotherapeutic composition targeting PRAME and/or PSMA or one or more epitopes of one or both antigens, wherein an effector T cell response is generated against at least one target antigen or one or more epitopes of one or both antigens. In some embodiments, the immunotherapeutic composition also targets at least one additional antigen associated with the tumor or one or more epitopes thereof.

In some embodiments, patients having a minimal baseline pre-existing immunoreactivity to PRAME and/or PSMA of less than a predetermined value, for example, about 0.2% of CD8⁺ T cells as determined by MHC multimer staining analysis are selected. In particular embodiments, patients having a baseline immunoreactivity to PRAME and/or PSMA of less than about 0.20%, about 0.15%, about 0.14%, about 0.13%, about 0.12%, about 0.11%, about 0.10%, about 0.09%, about 0.08%, about 0.07%, about 0.06%, about 0.05%, about 0.04%, about 0.03%, about 0.02%, or about 0.01% (% T cells stained out of all CD8+ T cells) as determined by MHC multimer analysis, or not distinguishable from control, are characterized as having no or minimal pre-existing immunoreactivity to the target antigen. A person of ordinary skill in the art, reading the instant application, would understand that the predetermined value against which the baseline immunoreactivity is compared can be chosen based on the specific assay that is used to evaluate the immunoreactivity.

In some embodiments, a patient with a PRAME and/or PSMA positive carcinoma and no, or minimal, pre-existing immunoreactivity to a targeted antigen or epitope, as measured for example by MHC multimer, and/or ELISPOT assay, and/or the like, is selected and an active immunotherapeutic targeting the PRAME and/or PSMA antigen or one or more epitopes thereof is administered. In some embodiments, the carcinoma is selected from melanoma including ocular melanoma, prostate cancer including hormone sensitive and hormone refractory prostate cancer, kidney cancer such as, for example, renal cell carcinoma, pancreatic, colorectal, and the like. In some embodiments, patients with these particular cancers are selected for treatment. In some embodiments, the active immunotherapeutic can also target additional antigens associated with the tumor.

A clinical benefit can include, for example, but is not limited to, tumor regression (partial or complete), stabilization of disease, prolonged survival, PSA velocity (doubling time) or expression level, and the like. For example, in some embodiments, the target antigen includes PRAME and/or PSMA and the patient shows an immune response to at least one of the target antigens, wherein the method includes co-administering an agent to reduce the immunosuppressive tumor microenvironment to promote a clinical benefit.

### Immunotherapeutic Compositions

Generally, any immunogen inducing an effector T cell response against the targeted antigens can be used, especially compositions suitable for intralymphatic delivery. Thus embodiments described herein relate to active immunotherapeutic compositions targeting an antigen, or one or more epitopes thereof. An antigen contemplated in embodiments disclosed herein, is one against which a response can be mounted by the immune system of a subject, having a malignant tumor for example, to attack a tumor thereby inhibiting its growth or shrinking or reducing or eliminating it, and hence treating or curing the disease. The antigen, in some instances, can be matched to the specific disease found in the subject being treated, to generate an effector T cell response (also referred to as a cell-mediated immune response). In some embodiments, the immune response is a CTL (a cytotoxic T lymphocyte) response, i.e. a cytotoxic reaction by the immune system that results in lysis of the target cells (e.g., the malignant tumor cells).

Antigens can include, but are not limited to, proteins, peptides, polypeptides and derivatives thereof, and can also be non-peptide macromolecules. In some embodiments, the antigen is a protein. In some embodiments, the antigen is a peptide. In some embodiments, the antigen is a polypeptide. In some embodiments, the antigen is a derivative of a protein, peptide or polypeptide. Thus, embodiments of the disclosure utilize peptide antigens of 8-15 amino acids in length, such as antigens of 8 amino acids in length, or antigens of 9 amino acids in length, or antigens of 10 amino acids in length, or antigens of 11 amino acids in length, or antigens of 12 amino acids in length, or antigens of 13 amino acids in length, or antigens of 14 amino acids in length, or antigens of 15 amino acids in length. Such a peptide can be an epitope of a larger antigen, i.e.it is a peptide having an amino acid sequence corresponding to the site on the larger molecule that is presented by MHC/HLA molecules and can be recognized by, for example, an antigen receptor or T-cell receptor. These smaller peptides are available to one of skill in the art and can be obtained by following the teachings of U.S. Pat. Nos. 5,747,269 and 5,698,396; and PCT Application Nos. PCT/EP95/02593 (Pub. No. WO/1996/001429) filed Jul. 4, 1995, and PCT/DE96/00351 (Pub. No. WO/1996/027008) filed Feb. 26, 1996, each of which is hereby incorporated by reference in its entirety. Additional approaches to epitope discovery are described in U.S. Pat. Nos. 6,037,135 and 6,861,234, each of which is incorporated herein by reference in its entirety.

Exemplary antigens include, PRAME, PSMA (prostate-specific membrane antigen), Melan A, and tyrosinase, SSX-2, NYESO-1, MAGE-1, MAGE-3, BAGE, GAGE-1, GAGE-2, CEA, RAGE, SCP-1, Hom/Me1-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CAM 17.1, NuMa, K-ras, β-Catenin, CDK4, Mum-1, p16, p15, 43-9F, 5T4, 791Tgp72, alpha-fetoprotein, β-HCG, BCA225, BTAA, CA 125, CA 15-3\CA 27.29\BCAA, CA 195, CA 242, CA-50, CAM43, CD68\KP1, CO-029, FGF-5, G250, Ga733\EpCAM, HTgp-175, M344, MA-50, MG7-Ag, MOV18, NB/70K, NY-CO-1, RCAS1, SDCCAG16, PLA2, TA-90\Mac-2 binding protein\cyclophilin C-associated protein, TAAL6, TAG72, TLP, and TPS. Each of the antigen names described herein is associated with a genetic locus. It will be apparent to those of ordinary skill in the art that there is genetic variation in the human population so that any particular reference sequence should be viewed as an example and naturally occurring variants found at the associated locus should be understood to be within the scope encompassed by the antigen name. In some embodiments, the variant can be recognized by a response induced or stimulated by the reference molecule. In some embodiments, the reference molecule can be recognized by a response induced or stimulated by the variant.

Embodiments of the disclosure relate to active immunotherapeutic compositions targeting an antigen(s) expressed by a tumor, or one or more epitopes thereof, for the treatment of cancer. Tumor-associated antigens (TuAAs) can be expressed by the cancer cell itself or associated with non-cancerous components of the tumor, such as tumor-associated neovasculature or other stroma. TuAAs can include antigens that are expressed on normal cells during fetal development when the immune system is immature and unable to respond, or they can be antigens that are normally present at extremely low levels on normal cells but are expressed at much higher levels on tumor cells. In some embodiments, a TuAA is an antigen associated with non-cancerous cells of the tumor, such as, for example, tumor neovasculature or other stromal cells within the tumor microenvironment. TuAA expression can be used to match a patient's cancer condition or type with an appropriate immunotherapeutic protocol or regimen.

In some embodiments, bivalent or multivalent therapeutic agents are used. Such bivalent or multivalent therapeutics can target more than one antigen on a tumor cell. In instances where more than a single antigen on a tumor cell is targeted, the effective concentration of antitumor therapeutic is increased accordingly. Attack on stroma associated with the tumor, such as vasculature, can increase the accessibility of the tumor cells to the agent(s) targeting them. Thus, even an antigen that is also expressed on some normal tissue can receive greater consideration as a target antigen if the other antigens to be targeted in a bi- or multivalent attack are not also expressed by that tissue. Moreover, by directing an immune response against more than a single antigen, if properly selected, the number of tumor cells that can be recognized is maximized. Further, some tumors lose expression of a TuAA following immunization, giving rise to a resistant population. If the immune response is directed against more than one TuAA it becomes much more difficult for a resistant tumor to arise because it must then simultaneously lose expression of each of the antigens in order to escape. Thus, in treating cancer with immunotherapy, it can be advantageous to use a combination of TuAAs both due to more complete coverage of the population of tumor cells, and because there will be less chance of tumor escape through loss of expression of the TuAAs. In preferred embodiments, this multivalent attack technique is employed when a tumor is positive for two, three, four or more TuAAs of the combination used.

In some embodiments, immunogenic peptides, corresponding to targeted epitope sequences of the antigen are contemplated herein and can include native sequence or cross-reactive analogue sequences. Exemplary analogues of Melan A, tyrosinase, PRAME and PSMA epitopes are disclosed in U.S. application Ser. No. 11/156,253 (Publication No. 20060063913), Ser. No. 11/155,929, (Publication No. 20060094661), Ser. No. 11/156,369 (Publication No. 20060057673), Ser. No. 11/454,300 (Publication No. US 2007-0060518 A1), Ser. No. 12/406,022, and U.S. Pat. Nos. 7,605,227; 7,511,118; and 7,511,119; each of which is hereby incorporated by reference in its entirety. “Cross-reactive analogue” as used herein can refer to a peptide including 1-3 amino acid substitutions, and/or one amino acid deletion or addition as compared to the native peptide sequence that induces effector function (e.g., cytolysis or cytokine secretion) distinguishable from background, from a CTL reactive with the native peptide. In various embodiments effector function is at least about 30%, about 50%, about 60%, about 70%, or about 80% of that induced by the native peptide. Alternatively cross reactivity can be demonstrated by the ability of the native peptide to induce effector function (e.g., cytolysis or cytokine secretion) distinguishable from background from a CTL generated by immunization with the analogue. In various embodiments effector function is at least 30%, 50%, 60%, 70%, or 80% of that induced by the analogue peptide.

In particular embodiments, examples of targeted antigens and/or an analogue thereof contemplated for use in the methods disclosed herein include PRAME₄₂₅₋₄₃₃, PSMA₂₈₈₋₂₉₇, Melan A₂₆₋₃₅, and Tyrosinase₃₆₉₋₃₇₇ disclosed in Table 1 and elsewhere herein.

TABLE 1
PARTIAL LISTING OF SEQ ID NOs

| SEQ ID NO | IDENTITY | SEQUENCE |
|---|---|---|
| 1 | Melan-A₂₆₋₃₅ | EAAGIGILTV |
| 2 | Melan-A₂₆₋₃₅ Analogue | ENvaAGIGILTV |
| 3 | Melan-A₂₆₋₃₅ Analogue | ELAGIGILTV |
| | A27L | |
| 4 | Tyrosinase369-377 | YMDGTMSQV |
| 5 | Tyrosinase369-377 | YMDGTMSQNva |
| | Analogue | |
| 6 | PRAME425-433 | SLLQHLIGL |
| 7 | PRAME425-433 Analogue | SNvaLQHLIGNle |
| 8 | PSMA288-297 | GLPSIPVHPI |
| 9 | PSMA288-297 Analogue | GLPSIPVHPV |

Melan A, also known as MART-1 (Melanoma Antigen Recognized by T cells), is a melanin biosynthetic protein also expressed at high levels in melanomas. Melan A/MART-1 is disclosed as a TuAA in U.S. Pat. Nos. 5,994,523; 5,874,560; and 5,620,886, each of which is hereby incorporated by reference in its entirety. In some embodiments disclosed herein, the targeted antigen is Melan A₂₆₋₃₅ and/or an analogue thereof disclosed in Table 1 and elsewhere herein. Examples of Melan A analogues are disclosed in U.S. Pat. No. 7,605,227 which is hereby incorporated by reference in its entirety. Melan-A analogs can include but are not limited to analogs of the sequence: E{A, L, Nva, or Nle}AGIGILT{V, Nva, or Nle} (SEQ ID NO:10); or Y{M, V, Nva, or Nle}DGTMSQ{V, Nva, or Nle} (SEQ ID NO:11); and wherein the sequence is not E{A or L}AGIGILTV (SEQ ID NO:12) or YMDGTMSQV (SEQ ID NO:4). The isolated peptide analogue of the invention may be selected from the group consisting of ELAGIGILTNva (SEQ ID NO:13), ENvaAGIGILTV (SEQ ID NO:2), YVDGTMSQNva (SEQ ID NO:14), YVDGTMSQV (SEQ ID NO:15) and YMDGTMSQNva (SEQ ID NO:5). In some embodiments, the analog is an analog consisting essentially of the amino acid sequence ENvaAGIGILTV (SEQ ID NO:2). In yet other embodiments the analog is an analog consisting essentially of the amino acid sequence YMDGTMSQNva (SEQ ID NO:5).

Tyrosinase, a melanin biosynthetic enzyme, is predominantly expressed in melanocytes with high levels often observed in melanomas. Tyrosinase is considered one of the most specific markers of melanocytic differentiation. It is also expressed in glial cells, which like melanocytes, develop from the neuroectoderm. Tyrosinase is thus also a useful TuAA for glioblastomas, including glioblastoma multiform. Further details of tyrosinase as a TuAA is disclosed in U.S. Pat. No. 5,747,271, which is hereby incorporated by reference in its entirety. In some embodiments disclosed herein, the targeted antigen is tyrosinase₃₆₉₋₃₇₇, and/or an analogue thereof disclosed in Table 1 and elsewhere herein.

PRAME, also known as MAPE, DAGE, and OIP4, is known in the art as a cancer-testis (CT) antigen. However, unlike many CT antigens, such as: MAGE, GAGE and BAGE, it is expressed in acute myeloid leukemias. PRAME as a TuAA is disclosed in U.S. Pat. No. 5,830,753, which is hereby incorporated by reference in its entirety. In some embodiments, the targeted antigen is PRAME_{425-433,} and/or an analogue thereof disclosed in Table 1 and elsewhere herein. Examples of PRAME analogues are disclosed in U.S. Pat. No. 7,511,119 which is hereby incorporated by reference in its entirety. PRAME analogs can include but are not limited to analogs including or consisting essentially of a sequence in which: P0 is X, XX, or XXX, wherein X specifies any amino acid or no amino acid; and P1 is S, K, F, Y, T, Orn, or Hse; and P2 is L, V, M, I, Nva, Nle, or Abu; and P3 is L, Nva, Nle or Abu; and P4 is Q; and P5 is H; and P6 is L, Nva, Nle, or Abu; and P7 is I; and P8 is G, A, S, or Sar; and PΩ at P9 is L, V, I, A, Nle, Nva, Abu, or L-NH₂; and PΩ+1 is X, XX, or XXX, wherein X specifies any amino acid or no amino acid; and wherein the sequence is not SLLQHLIGL (SEQ ID NO:6). A PRAME analog can include or consist essentially of the sequence: {K, F, Y, T, Orn, or Hse}LLQHLIGL (SEQ ID NO:16); or S{V, M, I, Nva, Nle, or Abu}LQHLIGL (SEQ ID NO:17); or SL{Nva, Nle or Abu}QHLIGL (SEQ ID NO:18); or SLLQH{Nva, Nle or Abu}IGL (SEQ ID NO:19); or SLLQHLI{A, S, or Sar}L (SEQ ID NO:20); or SLLQHLIG{V, I, A, Nle, Nva, Abu, or L-NH₂} (SEQ ID NO:21); or {F, Y, T, Orn, or Hse} {Nva, Nle, M, or I}LQHLIGL (SEQ ID NO:22); or S{Nva, Nle, or M}LQHLIG{Nva, Nle, or V} (SEQ ID NO:23); or {K, F, Y, T, Orn, or Hse}LLQHLIGV (SEQ ID NO:24); or {F or T}LLQHLIG{Nle} (SEQ ID NO:25); or {F or T} {Nva or M}LQHLIG{Nle} (SEQ ID NO:26). Also, a PRAME analog can include or consist essentially of the sequence: {F, Y, T, Orn, or Hse}LLQHLIGL (SEQ ID NO:27); or S{Nva, Nle, or M}LQHLIGL (SEQ ID NO:28); or SLLQHLIG{Nle, Nva, or L-NH₂} (SEQ ID NO:29); or SLLQH{Nva or Abu}IGL (SEQ ID NO:30); or S{Nva}LQHLIG{Nle} (SEQ ID NO:31); or {F or T} {L or Nva}LQHLIG{Nle} (SEQ ID NO:32). Further, the analog can include or consist essentially of the sequence: S{L or Nva}LQHLIG{Nle} (SEQ ID NO:33); or T{Nva}LQHLIG{Nle} (SEQ ID NO:34). The analog can include or consist essentially of the sequence S{Nva}LQHLIG{Nle} (SEQ ID NO:31).

Prostate-specific membrane antigen (PSMA) is found to be highly expressed in prostate cancer cells. However, PSMA expression is also noted in normal prostate epithelium and in the neovasculature of non-prostatic tumors. PSMA as an anti-neovasculature preparation is disclosed in U.S. Provisional Patent Application Ser. No. 60/274,063, and U.S. patent application Ser. No. 10/094,699 (Publication No. 20030046714) and Ser. No. 11/073,347 (Publication No. 20050260234); each of which is incorporated herein by reference in its entirety. PSMA as a TuAA is described in U.S. Pat. No. 5,538,866 incorporated herein by reference in its entirety. In some embodiments, the targeted antigen is PSMA₂₈₈₋₂₉₇ and/or an analogue thereof disclosed in Table 1 and elsewhere herein. Examples of PSMA analogues are disclosed in U.S. Pat. No 7,511,118 which is hereby incorporated by reference in its entirety. PSMA analogs can include but are not limited to analogs including or consisting essentially of a sequence in which: P0 is X, XX, or XXX, wherein X specifies any amino acid or no amino acid; and P1 is G, A, S, Abu, or Sar; and P2 is L, M, I, Q, V, Nva, Nle, or Abu; and P3 is P or W; and P4 is S; and P5 is I; and P6 is P; and P7 is V; and P8 is H; and P9 is P, A, L, S, or T; and PΩ at P10 is I, L, V, Nva, or Nle; and PΩ+1 is X, XX, or XXX, wherein X specifies any amino acid or no amino acid; and wherein the sequence is not GLPSIPVHPI (SEQ ID NO:8). The analog can include or consist essentially of the sequence: {S, Sar, or Abu}LPSIPVHPI (SEQ ID NO:35); or G{M or Nle}PSIPVHPI (SEQ ID NO:36); or G{L, I, Nva, or Nle}WSIPVHPI (SEQ ID NO:37); or GLWSIPVHP{Nva or V} (SEQ ID NO:38); or GLPSIPVH{A or S}I (SEQ ID NO:39); or GLPSIPVHP{V, L, Nva, or Nle} (SEQ ID NO:40); or G{Nle}PSIPVHP{Nva, or Nle} (SEQ ID NO:41); or G{Nva}PSIPVHP{Nva} (SEQ ID NO:42); or G{V, Nva, or Nle}PSIPVHPV (SEQ ID NO:43); or {Sar or Abu}LPSIPVHP{V or Nva} (SEQ ID NO:44); or A{V, I, Nva, or Nle}WSIPVHPI (SEQ ID NO:45); or AVPSIPVHP{V or Nva} (SEQ ID NO:46); or A{Nva}PSIPVHPV (SEQ ID NO:47); or ALWSIPVHP{V or Nva} (SEQ ID NO:48); or GVWSIPVHP{V or Nva} (SEQ ID NO:49); or G{Nva}WSIPVHPV (SEQ ID NO:50). Also, the analog can include or consist essentially of the sequence: {Abu}LPSIPVHPI (SEQ ID NO:51); or G{V, Nva, or Abu}PSIPVHPI (SEQ ID NO:52); or GLPSIPVHP{V or Nva} (SEQ ID NO:53); or GLWSIPVHP{I or Nva} (SEQ ID NO:54); or G{Nle}PSIPVHP{Nva} (SEQ ID NO:55); or G{Nle or Nva}PSIPVHPV (SEQ ID NO:56); or {A or Abu}LPSIPVHP{V or Nva} (SEQ ID NO:57); or G{Nva}WPSIPVHP{I or V} (SEQ ID NO:58); or A{Nva or Nle}WSIPVHPI (SEQ ID NO:59); or A{V or Nva}PSIPVHPV (SEQ ID NO:60). In particular, the analog can include or consist essentially of the sequence: {Abu}LPSIPVHPI (SEQ ID NO:51); or GLPSIPVHP{V or Nva} (SEQ ID NO:53); or GLWSIPVHPI (SEQ ID NO:61); or G{Nle}PSIPVHP{Nva} (SEQ ID NO:55). Preferably, the analog can include or consist essentially of the sequence: GLPSIPVHPV (SEQ ID NO:9).

In some embodiments, the immunotherapeutic regimen includes administration of two particular peptide analogues (E-PRA (SEQ ID NO:7) and E-PSM (SEQ ID NO:9)). As used herein, E-PRA refers to an analogue of PRAME₄₂₅₋₄₃₃, PRAME₄₂₅₋₄₃₃ L426Nva L433Nle (SEQ ID NO:7); E-PSM refers to an analogue of PSMA₂₈₈₋₂₉₇, PSMA₂₈₈₋₂₉₇ I297V (SEQ ID NO:9). These and other analogues are disclosed in U.S. patent application Ser. No. 11/156,369 (Pub. No. 20060057673), and U.S. Provisional Patent Application Ser. No. 60/691,889, both entitled “EPITOPE ANALOGS,” each of which is hereby incorporated by reference in its entirety.

In some embodiments, the immunotherapeutic regimen includes administration of two particular peptide analogues (E-MEL and E-TYR). As used herein, E-MEL refers to an analogue of Melan A_{26-35,} Melan A₂₆₋₃₅ A27Nva (SEQ ID NO:2); E-TYR refers to an analogue of tyrosinase₃₆₉₋₃₇₇, tyrosinase₃₆₉₋₃₇₇ V377Nva (SEQ ID NO:5). These and other analogues (including Melan A₂₆₋₃₅ A27L (SEQ ID NO:3)) are disclosed in U.S. patent application Ser. No. 11/156,369 (Pub. No. 20060057673), and U.S. Provisional Patent Application Ser. No. 60/691,889, both entitled “EPITOPE ANALOGS,” each of which is hereby incorporated by reference in its entirety.

Additional exemplary epitopes and epitope analogues are described in U.S. patent application Ser. No. 09/999,186, filed Nov. 7, 2001, entitled “METHODS OF COMMERCIALIZING AN ANTIGEN”; U.S. patent application Ser. No. 11/323,572 (Pub No. 20060165711); and U.S. patent application Ser. No. 11/323,520 (Pub. No. 20080124352), Ser. No. 10/117,937, (Pub. No. 20030220239 A1); Ser. No. 11/067,159 (Pub. No. 20050221440) and 11/067,064 (Pub. No. 20050142144), each filed Feb. 25, 2005; Ser. No. 10/657,022 (Pub. No. 20040180354) filed Sep. 5, 2003, and PCT Application Nos. PCT/US2003/027706, filed Jun. 17, 2004 (Pub. No. WO 04022709 A2) and PCT/US02/11101, filed Apr. 4, 2002 (Pub. No. WO 02081646), all “EPITOPE SEQUENCES”; and Ser. No. 10/292,413, (Pub. No. 20030228634 A1), 10/837,217 (Pub. No. 20040203051), and U.S. Pat. No. 7,232,682, each of which is hereby incorporated by reference in its entirety. Beneficial epitope selection principles for immunotherapeutics are disclosed in U.S. patent application Ser. Nos. 09/560,465 (filed on Apr. 28, 2000), 11/683,397 (Pub No. 20070269464), 10/026,066 (Pub. No. 20030215425 A1), 10/895,523 (Pub No. 2005-0130920), 10/896,325 (Pub No. 20070184062) and 10/005,905 (filed on Nov. 7, 2001), all entitled “EPITOPE SYNCHRONIZATION IN ANTIGEN PRESENTING CELLS”; 09/561,571 (filed on Apr. 28, 2000) entitled “EPITOPE CLUSTERS”; U.S. patent application Ser. Nos. 11/073,347 (Pub. No. 20050260234), 11/772,811 (Pub. No. 20090208537), and U.S. Pat. No. 7,252,824, each entitled “ANTI-NEOVASCULATURE PREPARATIONS FOR CANCER”; and U.S. patent application Ser. Nos. 10/117,937 (filed on Apr. 4, 2002; Pub. No. 20030220239 A1), and 10/657,022 (Pub. No. 20040180354 A1) and 12/194,478 (Pub. No. 20090285843), all entitled “EPITOPE SEQUENCES”; U.S. patent application Ser. No. 10/956,401 (Pub. No. 20050069982), and U.S. Pat. No. 6,861,234, both entitled “METHOD OF EPITOPE DISCOVERY”; each of which is hereby incorporated by reference in its entirety. It is also contemplated that new epitopes identified by the method disclosed in U.S. Pat. No. 6,861,234 entitled “METHOD OF EPITOPE DISCOVERY,” and U.S. patent application Ser. No. 10/026,066 (Pub. No. 20030215425) filed on Dec. 7, 2000 and entitled “EPITOPE SYNCHRONIZATION IN ANTIGEN PRESENTING CELLS,” (each of which is hereby incorporated by reference in its entirety) that is presently apparent or can be apparent in the future to one of ordinary skill in the art, are useful in embodiments disclosed herein.

Further embodiments relate to immunogenic compositions including a nucleic acid encoding an antigen or immunogenic fragment thereof. As discussed elsewhere herein, some methods utilize immunotherapeutic compositions for the treatment of cancer including plasmid(s) used in combination with synthetic peptide(s). The plasmid can be placed under the control of a promoter/enhancer sequence which allows for efficient transcription of messenger RNA for the polypeptide upon uptake by antigen presenting cells (APCs). Promoters that can be employed in embodiments disclosed herein are well known to one of ordinary skill in the art. Such promoters include, for example, viral and cellular promoters. Viral promoters can include, for example, but not limited to, the cytomegalovirus (CMV) promoter, the EF1 promoter, the major late promoter from adenovirus 2 and the SV40 promoter. Examples of cellular promoters include, for example, but are not limited to, the mouse metallothionein 1 promoter, elongation factor 1 alpha (EF1-alpha), MHC Class I and II promoter, and CD3 promoter (regarding T cell specific expression, long term). Other promoters that can be employed in the design of the vectors disclosed herein can be readily determined by the skilled artisan. Particular embodiments of the disclosure employ a promoter/enhancer sequence from cytomegalovirus (CMVp).

A polyadenylation signal can be provided at the 3′ end of the encoding sequence, for example a bovine growth hormone polyadenylation signal (BGH polyA), to increase messenger RNA stability and its translocation out of the nucleus and into the cytoplasm for translation. To facilitate plasmid transport into the nucleus after uptake, a nuclear import sequence (NIS) from simian virus 40 (SV40) can be inserted in the plasmid backbone. The plasmid design can also include immunostimulatory motifs, for example, a CpG immunostimulatory motif can be placed in the NIS sequence and/or the plasmid backbone. Immunostimulatory sequence (ISS) refers to an oligodeoxyribonucleotide containing an unmethlylated CpG sequence.

In some embodiments, the immunotherapeutic regimen includes a recombinant plasmid (pPRA-PSM). As used herein, pPRA-PSM refers to the plasmid RP12, (described in U.S. patent application Ser. No. 11/454,616 (Pub. No. 20070004662), entitled “METHODS AND COMPOSITIONS TO ELICIT MULTIVALENT IMMUNE RESPONSES AGAINST DOMINANT AND SUBDOMINANT EPITOPES, EXPRESSED ON CANCER CELLS AND TUMOR STROMA,” which is incorporated herein by reference in its entirety) which includes a recombinant DNA plasmid expressing PRAME₄₂₅₋₄₃₃ and PSMA₂₈₈₋₂₉₇ epitopes and/or analogues thereof (Table 1 and 2). The pPRA-PSM plasmid encodes the PRAME and PSMA epitopes in a manner that allows for their expression and presentation by pAPCs.

In some embodiments, the immunotherapeutic composition includes a pMEL-TYR plasmid. As used herein, pMEL-TYR refers to the plasmid pSEM, (described in detail and referred to as pMA2M in U.S. patent application Ser. No. 10/292,413 (Publication No. 20030228634), entitled “EXPRESSION VECTORS ENCODING EPITOPES OF TARGET ASSOCIATED ANTIGENS AND METHODS FOR THEIR DESIGN,” which is incorporated herein by reference) expressing the Melan A₂₆₋₃₅ A27L analogue and tyrosinase₃₆₉₋₃₇₇ epitopes (Table 1 and 2). It is understood that pMEL-TYR, pSEM and pMA2M are used interchangeably herein. The pMEL-TYR plasmid encodes the Melan A and tyrosinase epitopes in a manner that allows for their expression and presentation by pAPCs.

TABLE 2
PARTIAL LISTING OF SEQ ID NOs

| SEQ | | |
|---|---|---|
| ID NO | IDENTITY | SEQUENCE |
| 62 | pRP12 plasmid | KRSLLQHLIGLGDAAY |
| | liberation sequence | SLLQHLIGLISPEKEEQYIASL |
| | | LQHLIGLKRPSIKR |
| | | GLPSIPVHPV |
| 63 | pRP12 encoded | MNLLHETDSAVATARRPRWL |
| | immunogenic | CAGALVLAGGFFLLGFLFGWFI |
| | polypeptide | KSAQLAGAKGVILYSDPADYF |
| | | APGVKSYPDGWNLPGGGVQR |
| | | GNILNLNGAGDPLTPGYPANE |
| | | YAYRRGIAEAVGLPSIPVHPIA |
| | | LQSLLQHLIGLSNLTHVLYPVP |
| | | LESYEDIHGTLHLERLAYLHAR |
| | | LRELLCELGRPSMVWLSANPC |
| | | PHCGDRTFYDPEPILCPCFMPN |
| | | KRSLLQHLIGLGDAAYSLLQH |
| | | LIGLISPEKEEQYIASLLQHLIGL |
| | | KRPSIKRGLPSIPVHPV |
| 64 | pSEM plasmid | MLLAVLYCLELAGIGILTVYM |
| | liberation sequence | DGTMSQV |
| 65 | pSEM encoded | MLLAVLYCLELAGIGILTVYM |
| | immunogenic | DGTMSQVGILTVILGVLLLIGC |
| | polypeptide | WYCRRRNGYRALMDKSLHVG |
| | | TQCALTRRCPQEGFDHRDSKV |
| | | SLQEKNCEPV |

Thus in various embodiments, immunotherapeutic products including assemblages of immunogenic compositions can be used. Such assemblages can include 1, 2, or 3 plasmids as a set of individual compositions or a single composition can include two or more plasmids. Such assemblages can also include multiple peptides corresponding to the epitopes expressed by the plasmids. Similarly, they can be provided as compositions including individual or multiple peptides. In some embodiments, an priming/inducing/entraining plasmid or plasmids will be sold together with the corresponding boosting/amplifying peptides. In some embodiments, the multiple plasmids will be sold together, but without corresponding peptides. In still some embodiments, sets of corresponding peptides will be sold together without the plasmid, for example, for subsequent rounds of amplification of an entrained or primed response.

In further embodiments, each of the assemblages above include the peptides corresponding to (that is capable of boosting/amplifying the response to) the epitopes expressed by those plasmids. Some particular embodiments include an individual plasmid and one or both corresponding peptides. (Although the specific plasmids referred to herein are described as bivalent, they can also be amplified in a monovalent fashion).

In some embodiments, the clinical benefit can be enhanced or augmented. For example, in some embodiments, the immunotherapeutic regimen includes an agent that alters the tumor micro-environment so as to make it less immunosuppressive. As used herein, the tumor micro-environment refers to areas adjacent to and in between cancerous cells, encompassing extracellular matrix, vascular cells and other non-cancerous cells. In some embodiments, clinical benefit can be further improved—for example, in patients with less than a complete response, or in patients with visceral metastases, and the like—by combining the immunotherapeutic agent or composition with an agent that alters the tumor micro-environment so as to make it less immunosuppressive. Such agents include, for example, those useful to deplete T_{reg} cells or promote tumor inflammation and biological response modifiers (BRMs) capable of down-modulating or overcoming T_{reg} activity, including immunopotentiators, as discussed elsewhere herein, but are not limited to such. Exemplary T_{reg} depleting agents include, for example, IL-2 fusion molecule (denileukin diphtitox), anti-CD25 antibody, cyclosphosphamide, gemcitabine, fludarabine, doxorubicin, and the like. In some embodiments, agents that alter the tumor micro-environment so as to make it less immunosuppressive include small molecules, such as, for example: cyclophosphamide, fludarabine, select tyrosine kinase inhibitors, biomolecules such as LIGHT, TLR ligands, CpG molecules, antibodies against PD-1, CTLA-4, TGFbeta, IL-10, and the like. An exemplary method including administration of agents to deplete Treg cells or promote tumor inflammation can be found in U.S. patent application Ser. Nos. 60/831,256, filed on Jul. 14, 2006; 60/863,332, filed on Oct. 27, 2006; 11/879,078 (Pub. No. 2008-0014211), filed on Jul. 14, 2007; and PCT Application Publication No. WO/2008/008541, filed on Jul. 14, 2007; all entitled “METHODS TO ELICIT, ENHANCE AND SUSTAIN IMMUNE RESPONSES AGAINST MHC CLASS I-RESTRICTED EPITOPES, FOR PROPHYLACTIC AND THERAPEUTIC PURPOSES,” each of which is incorporated herein by reference in its entirety. In some embodiments biological response modifiers (BRMs) capable of down-modulating or overcoming T_{reg} activity are administered.

In some embodiments, an immunopotentiator is administered with at least one component of the immunotherapeutic regimen. The immunopotentiator can be an immunomodulatory molecule, such as, but not limited to, TLR ligands, endocytic-Pattern Recognition Receptor (PRR) ligands, and the like. Thus, in some embodiments, clinical benefit can be further improved—for example, in patients with less than a complete response, or in patients with visceral metastases, and the like—by combining at least one component of the immunotherapeutic regimen with an immunopotentiator or a biological response modifier. In some embodiments, the response is assessed following two therapeutic cycles of treatment. Responses can be determined, for example, by: an increase in tetramer (MHC-multimer) positive cells, an increase in cytokine secretion (for example, secretion of a granzyme or gamma-interferon), or an increase in the proportions of effector or effector-memory T cells. Assessment of response can be based on assaying T cells in peripheral blood or recovered in tumor biopsies.

### Immunopotentiators

In some embodiments, the immunotherapeutic regimen includes an agent that alters the tumor micro-environment so as to make it less immunosuppressive. For example, in some embodiments, clinical benefit can be further improved—for example, in patients with less than a complete response, or in patients with visceral metastases, and the like—by combining the immunotherapeutic agent/product with an agent that alters the tumor micro-environment so as to make it less immunosuppressive. Example of such agents include, but are not limited to, small molecules, such as, cyclophosphamide, fludarabine, select tyrosine kinase inhibitors, and the like; biomolecules, Toll-like receptor (TLR) ligands (e.g., CpG motifs, antibodies against PD-1, CTLA-4, TGFbeta, IL-10, and the like; and/or biomolecules, such as LIGHT, and the like.

Immunopotentiators contemplated as useful in embodiments disclosed herein, include, for example and in a non-limiting manner: agents that are involved in the control of an immune response such as cytokines, chemokines, co-stimulatory molecules, transcription factors, and signal transduction elements; agents that are involved in antigen processing and presentation; agents that are involved in regulating the apoptotic pathway, or agents that are involved in gene control or silencing, such as DNA methylating enzymes, chromatin controlling molecules, RNA regulating molecules, or the like are included.

In other aspects of the disclosure, immunpotentiators can include, for example and in a non-limiting manner, molecules that trigger cytokine or chemokine production, such as peptidoglycans, LPS or analogues therefrom, unmethylated CpG oligodeoxynuclotides (CpG ODNs); dsRNAs such as bacterial dsDNA (which contains CpG motifs) and synthetic dsRNA (polyI:C) on APC and innate immune cells that bind to TLR9 and TLR3, respectively.

One class of immunopotentiators considered useful in embodiments disclosed herein, includes small organic natural or synthetic molecules, which exert immune modulating effects by stimulating pathways of innate immunity. It has been shown that macrophages, dendritic and other cells carry so-called Toll-like receptors (TLRs), which recognize pathogen-associated molecular patterns (PAMPs) on micro-organisms (Thoma-Uszynski, S. et al., Science 291:1544-1547, 2001; Akira, S., Curr. Opin. Immunol. 15: 5-11, 2003; each of which is incorporated herein by reference in its entirety). Further contemplated, are small molecules that bind to TLRs, such as the purely synthetic anti-viral imidazoquinolines, e.g., imiquimod and resiquimod, that have been found to stimulate the cellular path of immunity by binding the TLRs 7 and 8 (Hemmi, H. et al., Nat Immunol 3:196-200, 2002; Dummer, R. et al., Dermatology 207:116-118, 2003; each of which is incorporated herein by reference in its entirety). Immunopotentiators can further include immunopotentiating adjuvants that activate pAPC or T cells including, for example: endocytic-Pattern Recognition Receptor (PRR) ligands, quillaja saponins, tucaresol and the like.

Immunopotentiators can interact directly with receptors that detect microbial components. In some embodiments, additional therapeutic molecules include, but are not limited to, transcription factors such as T-bet, STAT-1 STAT-4 and STAT-6. Additionally, molecules that act downstream in the signaling pathway can also be used. In some embodiments, the targeted molecules can include TLR and its downstream signaling molecules such as, for example and not limited to, MyD88, NFκ-B and the like. Cytokines are also contemplated for use in embodiments disclosed herein, such as, for example but not limited to, interferons (e.g, .IFN-gamma), IL-2, IL-10, IL-12, IL-18 and TGF-beta GM-CSF, flt3 ligand (flt3L), TNF-alpha, and the like. Costimulatory factors such as CD40 B7-1 and B7-2 are also contemplated as useful herein. Antibodies that bind to co-stimulatory molecules (anti-CD40, CTLA-4, anti-OX40, and the like) are also contemplated as useful in embodiments of the disclosure. In some embodiments, checkpoint proteins such as, for example but not limited to, FOXp3, B7-like molecules, LAG-3 ligands and such molecules are also contemplated as useful in some embodiments. In some embodiments, chromatin controlling molecules and RNA regulating molecules are also contemplated. Proteins present in the antigen processing and presentation pathway such as, for example but not limited to, HLA and TAPs (Transporters associated with Antigen Processing-1 and -2 (TAP1 and TAP2)), immune or standard proteasome, beta-2-microglobulin, and MHC class I or II molecules are also contemplated for use in some embodiments. Dendritic cell activation suppressor SOCS1 and proteins in the DNA methylation pathway such as DMNT1 are also contemplated as useful in embodiments disclosed herein. Proteins present in the apoptotic pathway are also contemplated as useful in embodiments disclosed herein. Additionally, chemokines, such as, for example, but not limited to, IL-8, MIP-3-alpha, MIP-1-beta, MCP-1, MCP-3, RANTES, and the like can also be employed in embodiments disclosed herein. In some embodiments, additional therapeutic molecules involved in promoting or maintaining T cells and/or expression of differentiation antigens (for example melanoma differentiation antigens) are contemplated herein for use with the active immunotherapeutic agent disclosed. Such therapeutic molecules include in some embodiments, inhibitors of BRAF (serine/threonine kinase B-Raf), for example, PLX4720 (see also WO2007/002325 disclosed herein for all it contains relating to BRAF inhibitors) which can promote T cell expansion or persistence, increase differentiation antigen expression levels and promote T cell activity against the cancer. In some embodiments, inhibition of the MAPK pathway with MAPK/extracellular signal-regulated kinase (MEK) inhibitors are also contemplated.

### Immunotherapy Approaches

Ultimately any immunotherapeutic regimen that generates an effector T cell response against the targeted antigen(s) or epitopes can be used in the disclosed methods. Those including a step for intralymphatic administration of an immunogen are particularly suitable. Prime-boost type protocols are also considered advantageous. One “prime-boost” protocol is discussed in U.S. Pat. No. 6,663,871 entitled “Methods and reagents for vaccination which generate a CD8 T cell immune response,” which is hereby incorporated by reference in its entirety. The exemplary immunotherapeutic regimens applied in the methods disclosed herein are based on preclinical models utilizing an initial composition that establishes or entrains the immune response and a second composition that intensifies, augments or enhances the response to effective levels. In some embodiments, the first and second compositions are in forms that are the same, and in some embodiments, the first and second compositions are in forms that are different. An “entraining” immunogen as contemplated in embodiments disclosed herein includes, in many embodiments, an induction that confers particular stability on the immune profile of the induced lineage of T cells. Although not all features of the preclinical model have been observed in clinical use in humans, it is the inventors' understanding that the priming/inducing/entraining doses can be important in avoiding the tolerogenic effect that can otherwise result from the repeated administration of peptide as used in the boosting doses. While clinical responses utilizing a plasmid prime-peptide boost regimen have been shown to be superior to those obtained following immunization with plasmid alone (Tagawa et al., “Phase I Study of Intranodal Delivery of a Plasmid DNA Vaccine for Patients with Stage IV Melanoma” Cancer, Jul. 1, 2003, Vol. 98, Number 1, which is hereby incorporated by reference in its entirety.), prime-boost protocols utilizing a single form of immunogen—e.g., protein, polypeptide, peptide, plasmid, RNA, viral vector, and the like—can also be used in the methods disclosed herein.

In some embodiments, the immunotherapeutic regimen can involve a prime-boost protocol or induce-and-amplify protocol. In some embodiments, the immunotherapeutic regimen can involve an entrain-and-amplify protocol. Thus, in some embodiments, the active immunotherapeutic regimen includes a plasmid and one or more peptides or analogues thereof, which can be administered in treating a cancer in a subject using any such regimens/protocols (as disclosed in U.S. patent application Ser. No. 11/879,078 (Pub. No. 2008-0014211) and U.S. patent application Ser. No. 10/871,707 (Pub. No. 2005-0079152), U.S. patent application Ser. No. 11/323,572 (Pub. No. 2006-0165711) each entitled “METHOD TO ELICIT, ENHANCE AND SUSTAIN IMMUNE RESPONSES AGAINST MHC CLASS-I RESTRICTED EPITOPES, FOR PROPHYLACTIC OR THERAPEUTIC PURPOSES,” each of which is hereby incorporated by reference in its entirety). Other immunization protocols, for example, using plasmid for other than the initiation dose(s), relying on plasmid or peptide alone, or utilizing other types of boosting/amplifying reagents are not excluded from the scope of the disclosure. Some embodiments encompass bi- or multivalent plasmids expressing more than one target antigen, or one or more epitopes thereof.

Some embodiments use an entrain-and-amplify therapeutic regimen to achieve a multivalent attack, offering the advantage of increasing the sensitivity of the tumor to attack. If more than a single antigen on a tumor cell is targeted, the effective concentration of antitumor agent is increased accordingly. Attack on stroma associated with the tumor, such as vasculature, can also increase the accessibility of the tumor cells to the agent(s) targeting them. Thus, even an antigen that is also expressed on some normal tissue can receive greater consideration as a target antigen if the other antigens to be targeted in a multivalent attack are not also expressed by that tissue.

In some embodiments, exemplary immunotherapeutic regimens applied to the patient selected according to the methods disclosed herein involve priming with a plasmid such as, for example, pPRA-PSM or pMEL-TYR, and boosting with the respective peptide analogues, e.g., E-PRA and E-PSM, or E-MEL and E-TYR, respectively.

In some embodiments, the methods can include, applying a regimen that includes, for example, 1-6 priming/inducing/entraining doses. In some embodiments, the method can include administering a plurality of priming/inducing/entraining doses, wherein said doses are administered over a course of one to about seven days. The priming/inducing/entraining doses, boosting/amplifying doses, or priming/inducing/entraining and boosting/amplifying doses can be delivered in multiple pairs of injections, wherein a first member of a pair can be administered within about 4 days of a second member of the pair, and wherein an interval between first members of different pairs can be at least about 14 days. An interval between a last priming/inducing/entraining dose and a first boosting/amplifying dose can be between about 7 and about 100 days, for example.

As contemplated in the instant disclosure, the term “boosting/amplifying” or “boost/amplification,” as of a T cell response, includes, in many embodiments, a process for increasing the number of cells, the number of activated cells, the level of activity, rate of proliferation, or similar parameter of T cells involved in a specific response. In some embodiments, the immunotherapeutic regimen or protocol can be adjusted based on the responsiveness to induction or amplification phases and variation in antigen expression. For example, repeated priming or inducing or entraining doses can be administered until a detectable response is obtained before administering boosting or amplifying dose(s), rather than boosting or amplifying after some set number of priming/inducing/entraining doses. Similarly, scheduled boosting/amplifying or maintenance doses of peptide can be discontinued if their effectiveness wanes, antigen-specific regulatory T cell numbers rise, or some other evidence of tolerization is observed, and further priming/inducing/entraining doses can be administered before resuming boosting/amplifying doses. The integration of diagnostic techniques to assess and monitor immune responsiveness with methods of immunization is discussed more fully in Provisional U.S. patent application Ser. No. 60/580,964, which was filed on Jun. 17, 2004 and U.S. patent application Ser. No. 11/155,928 (Pub. No. 20050287068), filed Jun. 17, 2005, both entitled “IMPROVED EFFICACY OF ACTIVE IMMUNOTHERAPY BY INTEGRATING DIAGNOSTIC WITH THERAPEUTIC METHODS,” each of which is hereby incorporated by reference in its entirety.

In some embodiments, a vector composition is injected into the inguinal lymph node followed by subsequent administration of a peptide antigen as a bolus. In some embodiments, one or more priming/inducing/entraining composition(s) and one or more boosting/amplifying composition(s) antigen are administered intralymphatically. In some embodiments, one or more priming/inducing/entraining composition(s) or one or more boosting/amplifying composition(s) antigen are administered intralymphatically. For example, in some embodiments, one or more boosting/amplifying composition(s) antigen are administered intralymphatically. In some embodiments, one or more components can be delivered by infusion, generally over several hours to several days. For example, in some embodiments, the immunotherapeutic regimen or protocol calls for injection or infusion into one or more lymph nodes, starting with a number of administrations (e.g., 1 to 10, or more, 2 to 8, 3 to 6, 4 or 5 etc.) of recombinant DNA (dose range of about 0.001 to about 10 mg/kg, about 0.005 to about 5 mg/kg, such as about 0.01 to about 1 mg/kg, such as about 0.1 to about 0.5 mg/kg) followed by one or more, e.g., 2 or 3 or 4 or 5 or 6 or 7 or 8 or 9 or 10 administrations of peptide, preferably in an immunologically inert vehicle or formulation (dose range of about 1 mg/kg to about 10 mg/kg, preferred about 0.005 to about 5 mg/kg, such as about 0.01 to about 1 mg/kg, such as about 0.1 to about 0.5 mg/kg). Because dose does not necessarily scale linearly with the size of the subject, doses for humans can tend toward the lower, and doses for mice can tend toward the higher, portions of these ranges. In some embodiments, the preferred concentration of plasmid (or an effective amount of a plasmid) and peptide (or an effective amount of peptide) upon injection is generally about 0.1 mg/ml to about 10 mg/ml, such as about 0.5 to about 5 mg/ml, such as about 1 to about 2.5 mg/ml, or about 1 mg/ml, generally irrespective of the size or species of the subject. However, particularly potent peptides can have optimum concentrations (effective amounts) toward the low end of this range, for example, between about 1 and about 100 μg/ml, such as between about 10 and about 50 μg/ml, or between about 20 and about 60 μg/ml, or between about 30 and about 70 μg/ml, or between about 40 and about 80 μg/ml, or between about 50 and about 90 μg/ml.

The time between the last priming/inducing/entraining dose of DNA and the first boosting/amplifying dose of peptide is not critical. In some embodiments, the time between priming/inducing/entraining and boosting/amplifying is about 7 days or more, such as about 2 weeks or about 3 weeks or about 1 month, and can exceed several months. The multiplicity of injections of the priming/inducing/entraining doses and/or the boosting/amplifying doses can be reduced by substituting infusions lasting several days (preferred 2-7 days, such as 2 days or 3 days or 4 days or 5 days or 6 days or 7 days). It can be advantageous to initiate the infusion with a bolus of material similar to what might be given as an injection, followed by a slow infusion (about 24 to about 12000 microliters/day to deliver about 25 to about 2500 micrograms/day for DNA, about 0.1 to about 10,000 micrograms/day for peptide). This can be accomplished manually or through the use of a programmable pump, such as an insulin pump. Such pumps are known in the art and enable periodic spikes and other dosage profiles, which can be desirable in some embodiments.

### Other Therapies

In some embodiments, the immunotherapeutic regimen disclosed herein can be integrated with other traditional cancer therapies including, but not limiting to, surgery, radiation, chemotherapy, hormonal therapy and the like. In some embodiments, the immunotherapeutic regimen disclosed herein can be used in an adjuvant or neoadjuvant setting or treatment protocol to enhance or augment the clinical benefit. In some embodiments, the immunotherapeutic regimen can be incorporated into standard oncology therapy paradigms such as, adjunctive or consolidation therapy, involving surgery, radiation, chemotherapy, biotherapy, gene therapy, or hormonal therapy, and the like to enhance or augment the clinical benefit.

As used herein, “neoadjuvant setting treatment protocol” or “neoadjuvant therapy” refers to therapy or treatment prior to surgery or other subsequent therapy. That is, at least one therapeutic cycle of the immunotherapeutic regimen described herein is completed prior to a tumor ablative treatment such as, for example, but not limited to, surgery, radiation, or direct chemotherapy. In various embodiments, the tumor ablative treatment can be administered within days or two weeks of the completion of the therapeutic cycle as determined by the skilled practitioner. Thus, the use of the immunotherapeutic regimen in a neoadjuvant therapeutic setting can increase the rate of complete and partial remission in patients, decrease the rate of relapse, and/or increase median disease free survival thereby improving the clinical benefit.

In some embodiments, the immunotherapeutic regimen can be used in an adjuvant setting to increase the likelihood of a cure. That is, the cancer can be put into complete remission by a tumor ablative treatment such as, for example, but not limited to, surgical removal, irradiation, or chemotherapy with doses that are directly cytotoxic to the cancer cells, and the like. The immunotherapeutic regimen is subsequently administered, resulting in a decreased rate of relapse and increased interval of disease-free survival. The immunotherapeutic regimen can be administered within hours, days, or weeks of the completion of the initial treatment.

In some embodiments, the immunotherapeutic regimen can be used as consolidation therapy. This resembles the adjuvant setting described above except that complete remission is not necessarily attained. In this setting, the immunotherapeutic regimen improves or increases the time to progression and progression-free survival (in the case of partial remission), and decreases the rate or time to relapse (in the case of complete remission).

In some embodiments, the immunotherapeutic regimen can be used as adjunctive therapy, that is, in further combination with a tumor ablative treatment to increase that treatment's efficacy. In contrast to adjuvant therapy as described above in which the immunotherapeutic regimen is not initiated until the primary treatment is complete, here the two treatments are used together to increase the rate of response or disease control rate (that is of partial or complete remission). The actual schedule of the two treatments can be similar to those above, but therapeutic cycles of the immunotherapeutic regimen can be alternated with rounds of the primary treatment such as chemotherapy or radiation. In some embodiments, surgery can be carried out during the time interval of a therapeutic cycle of the immunotherapeutic regimen, for example in the interval between the priming (induction/entraining) and boost (amplification) stages or in an interval between courses of the amplification composition.

In exemplary embodiments, in a patient with skin or subcutaneous metastases, but not yet visceral metastases, the immunotherapy is used to stabilize the disease, or reduce or ablate tumor burden at the site of origin and/or in the lymphatic system, and the skin or subcutaneous tumors are surgically resected.

In exemplary embodiments, in a patient with skin or subcutaneous metastases, but not yet visceral metastases, the immunotherapy is used to stabilize the disease, or reduce or ablate tumor burden in the lymphatic system, prior to resection of the skin or subcutaneous tumors. Thus the immunotherapeutic regimen can be in a neoadjuvant setting (vaccine first, standard of care second) to stabilize the disease prior to tumor removal leading to long term remission. Exemplary cancers susceptible to such an approach include kidney carcinomas and melanomas.

For some cancers, for example melanoma, the typical course of treatment includes surgical resection of tumor at the site of origin. Such a course of treatment is also applicable to a variety of solid tumors particularly in, but not necessarily limited to, earlier stages of the disease. Examples include prostate and kidney cancer. Thus, in particular embodiments the immunotherapeutic regimen is applied in an adjuvant setting or as consolidation therapy, depending on the degree of spread of the disease that had already occurred.

### Methods of Delivering Compositions

In some embodiments, the disclosure relates to methods of administering the one or more therapeutic agents of the immunotherapeutic regimen disclosed herein. Such methods can include, without limitation: intralymphatic, intranodal, perinodal, oral, transdermal, intravenous, intradermal, intramuscular, intraperitoneal, mucosal administration, and the like. Administration can be in any manner compatible with the dosage formulation and in such amount as will be therapeutically effective. An effective amount or dose of an immunogenic composition of the disclosure is that amount found to provide a desired response in the subject to be treated. A particularly useful method of vaccine delivery to elicit a CTL response is disclosed in U.S. Pat. Nos. 6,994,851, 6,977,074, 7,364,729, and application Ser. Nos. 11/418,497 (Pub. No. 20090035252) and 11/418,397, each entitled “A METHOD OF INDUCING A CTL RESPONSE,” and application Ser. No. 12/070,156 (Pub. No. 20080199458), entitled “A METHOD OF ENHANCING A T-CELL RESPONSE,” each of which is incorporated by reference in its entirety.

In some embodiments, the compositions disclosed herein are administered directly to the lymphatic system or lymphatic organ. In particular embodiments, this is to a lymph node. Afferent lymph vessels can be similarly utilized. Choice of lymph node is not critical. Inguinal lymph nodes can be utilized for their size and accessibility, but axillary and cervical nodes and tonsils can be similarly advantageous. Administration to a single lymph node can be sufficient to induce or amplify an immune response. In some embodiments, administration to multiple nodes can increase the reliability and magnitude of the response. For embodiments promoting a multivalent response and in which multiple boosting/amplifying peptides are therefore used, each peptide can be administered to any lymph node on any occasion. Thus, one peptide can be administered to the right inguinal lymph node and a second peptide to the left inguinal lymph node at the same time, for example. Additional peptides can be administered to other lymph nodes even if they are not sites of induction, as it is not essential that initiating and boosting/amplifying doses be administered to the same site, due to migration of T lymphocytes. Alternatively, additional peptides can be administered a few days later, for example, to the same lymph nodes used for the previously administered boosting/amplifying peptides as the time interval between induction and amplification generally is not a crucial parameter. Thus, in some embodiments, the time interval can be greater than about a week. Segregation of administration of boosting/amplifying peptides is generally of less importance if their MHC-binding affinities are similar, but can grow in importance as the affinities become more disparate. Incompatible formulations of various peptides can also make segregated administration useful.

To introduce the components of the immunotherapeutic composition disclosed herein into the lymphatic system or lymphatic organ of the patient, each of the components is directed to a lymph vessel, lymph node, the spleen, or other appropriate portion of the lymphatic system. In some embodiments, each component is administered as a bolus. In some embodiments, one or more components are delivered by infusion, generally over several hours to several days. In particular embodiments, the immunotherapeutic compositions are directed to a lymph node such as an inguinal or axillary node by inserting a catheter or needle to the node and maintaining the catheter or needle throughout the delivery. Suitable needles or catheters are available made of metal or plastic (e.g., polyurethane, polyvinyl chloride (PVC), TEFLON, polyethylene, and the like). In inserting the catheter or needle into the inguinal node for example, the inguinal node is punctured under ultrasonographic control using a Vialon™ Insyte W.™. cannula and catheter of 24G¾ (Becton Dickinson, USA) which is fixed using Tegaderm transparent dressing (Tegaderm™, St. Paul, Minn., USA). An experienced radiologist generally does this procedure. The location of the catheter tip inside the inguinal lymph node is confirmed by injection of a minimal volume of saline, which immediately and visibly increases the size of the lymph node. The latter procedure allows confirmation that the tip is inside the node. This procedure can be performed to ensure that the tip does not slip out of the lymph node and can be repeated on various days after implantation of the catheter. In the event that the tip does slip out of location inside the lymph node, a new catheter can be implanted.

Various parameters can be taken into account in delivering or administering an immunogenic composition to a subject. In addition, a dosage regimen and immunization schedule can be employed. Generally the amount of the components in the immunotherapeutic composition will vary from patient to patient and from antigen to antigen, depending on such factors as: the activity of the antigen in inducing a response; the flow rate of the lymph through the patient's system; the weight and age of the subject; the type of disease and/or condition being treated; the severity of the disease or condition; previous or concurrent therapeutic interventions; the capacity of the individual's immune system to synthesize antibodies; the degree of protection desired; the manner of administration and the like, all of which can be readily determined by the practitioner.

In general, if being delivered by infusion the immunotherapeutic compositions described herein can be delivered at a rate of from about 1 to about 500 microliters/hour or about 24 to about 12,000 microliters/day. The concentration of the immunotherapeutic composition is such that about 0.1 micrograms to about 10,000 micrograms of the therapeutic composition will be delivered during a 24 hour period. The flow rate is based on the knowledge that, in each minute, approximately about 100 to about 1,000 microliters of lymph fluid flows through an adult inguinal lymph node. An objective is to maximize local concentration of vaccine formulation in the lymph system. A certain amount of empirical investigation on patients is conducted to determine the most efficacious level or optimal level of infusion for a given immunotherapeutic in humans.

In some embodiments, the immunogenic compositions described herein can be administered as a number of sequential doses. Such doses can be 2, 3, 4, or more doses as is needed to obtain the appropriate immune response. In some embodiments, it is contemplated that the doses of the immunogenic composition can be administered within about seconds or minutes of each other into the right or left inguinal lymph nodes. For example, the plasmid (prime) can first be injected into the right lymph node followed within seconds or minutes by a second plasmid into the right or left inguinal lymph nodes. In some embodiments, the combination of one or more plasmids expressing one or more immunogens can be administered. In some embodiments, the subsequent injection following the first injection into the lymph node can be within about 1, 2, 3, 4, 5, 6, 7, 8, 9, 10 or more minutes but not greater than about 30, 40, 50, or 60 minutes after the first injection. Similar considerations apply to the administration of two peptides individually to the right and left lymph nodes. It can be desirable to administer the doses of the immunogenic composition at an interval of days, for example, where 1, 2, 3, 4, 5, 6, or 7, or more days elapse between subsequent administrations. In some embodiments, it can be desirable for subsequent administration(s) of the compositions to be administered via bilateral inguinal lymph node injection within about 1, 2, 3, or more weeks or within about 1, 2, 3, or more months following the initial dose administration.

### Kits

In some embodiments, all or a subset of the components of the immunotherapeutic compositions disclosed herein can be packaged or assembled together in a kit. In some embodiments, the therapeutic proteins, peptides, polypeptides, epitopes or nucleic acid encoding such can be packaged together, or as single molecules or as a set of molecules. Alternatively, the compositions disclosed herein can be packaged and sold individually along with instructions, in printed form or on machine-readable media, describing how they can be used in conjunction with each other as disclosed herein, for use as a therapeutic.

In some embodiments, the kit can contain, in a suitable container means, one or more therapeutic agents and instructions for employing the methods disclosed herein. In some embodiments, the kit can have a single container means, and/or it can have distinct container means for additional compounds such as an immunological or therapeutic effective formulation of one or more therapeutic agents for treating a disease or condition due to, for example, a proliferative disease such as cancer.

Where the components of the kit are provided in one or more liquid solutions, the liquid solution is an aqueous solution, with a sterile aqueous solution being particularly preferred. The compositions can also be formulated as a deliverable and/or injectable composition. In such embodiments, the container means can itself be a syringe, pipette, and/or other such apparatus, from which the formulation can be delivered or injected into a subject, and/or even applied to and/or mixed with the other components of the kit. In some embodiments, the components of the kit can be provided as dried powder(s). When components (e.g., reagents) are provided as a dry powder, the powder can be reconstituted by the addition of a suitable solvent. It is envisioned that the solvent can also be provided in another container means. In some embodiments, one or more components of the kit can be provided as a liquid solution or a dry powder.

In some embodiments, the plasmid can be sold together with the therapeutic protein, peptide, epitope or nucleic acid encoding such. In some embodiments, sets of therapeutic proteins, peptides, epitopes or nucleic acids encoding such can be sold together without the plasmid. In some embodiments, each of the components including the immunotherapeutic regimen disclosed herein can be sold separately.

The container means will generally include at least one vial, test tube, flask, bottle, syringe and/or other container means, into which the one or more therapeutic agents can be placed. The kit can also include a second container means for containing a sterile, pharmaceutically acceptable buffer and/or other diluent. In some embodiments, the kit can also include a means for containing the materials for practicing the methods disclosed herein, and any other reagent containers in close confinement for commercial sale. Such containers can include, for example, injection or blow-molded plastic containers into which the desired vials are retained. Irrespective of the number or type of containers, the kit(s) described herein can also include, or be packaged with, an instrument for assisting with the injection/administration of the one or more therapeutic agents within the body of a subject. Such an instrument can be, for example, but not limited to, a syringe, pump and/or any such medically approved delivery vehicle.

Thus, in some embodiments, priming/entraining/inducing and boosting/amplifying compositions targeting a single epitope, or set of epitopes, can be packaged together. In other instances, multiple priming/entraining/inducing compositions can be assembled in one kit and the corresponding boosting/amplifying compositions assembled in another kit. Alternatively, compositions can be packaged and sold individually along with instructions, in printed form or on machine-readable media, describing how they can be used in conjunction with each other to achieve the beneficial results of the indicated immunization protocol or immunotherapeutic regimen. Further variations will be apparent to one of skill in the art. The use of various packaging schemes including less than all of the compositions that might be employed in a particular protocol or regimen facilitates the personalization of the treatment, for example, based on tumor antigen expression, or observed response to the immunotherapeutic or its various components, as described in U.S. application Ser. Nos. 11/155,288 (Pub. No 20060008468) and 11/323,964 (Pub. No. 2006-159689), each entitled “COMBINATIONS OF TUMOR-ASSOCIATED ANTIGENS IN DIAGNOSTICS FOR VARIOUS TYPES OF CANCERS,” and U.S. patent application Ser. No. 11/155,928 (Pub. No. 20050287068), entitled “IMPROVED EFFICACY OF ACTIVE IMMUNOTHERAPY BY INTEGRATING DIAGNOSTIC WITH THERAPEUTIC METHODS,” each of which is hereby incorporated by reference in its entirety.

In addition to those already disclosed in this application, the following applications are hereby expressly incorporated by reference in their entireties. U.S. Provisional Application Ser. Nos. 61/279,621 filed Oct. 23, 2009; 61/254,657 filed Oct. 24, 2009; and 61/255,850 filed Oct. 28, 2009, each entitled “CANCER IMMUNOTHERAPY AND METHOD OF TREATMENT,” each relate to improved strategies for the design and practice of generating an effector T cell response against a target antigen in a subject, and each is hereby expressly incorporated by reference in its entirety. Useful methods for using the disclosed analogs in inducing, entraining, maintaining, modulating and amplifying class I MHC-restricted T cell responses, and particularly effector and memory CTL responses to antigen, are described in U.S. Pat. Nos. 6,994,851, 6,977,074, and 7,364,729, and U.S. patent application Ser. No. 10/871,707 (Pub. No. 2005-0079152) and U.S. patent application Ser. No. 11/323,572 (Pub. No. 2006-0165711), entitled “METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSE.” The analogs can also be used in research to obtain further optimized analogs. Numerous housekeeping epitopes are provided in U.S. application Ser. Nos. 10/117,937 (Pub. No. 2003-0220239 A1), and 10/657,022 (Pub. No. 2004-0180354), and in PCT Application No. PCT/US2003/027706 (Pub. No. WO 04/022709A2; and U.S. Provisional Application Nos. 60/282,211, filed on Apr. 6, 2001; 60/337,017, filed on Nov. 7, 2001; 60/363,210 filed on Mar. 7, 2002; and 60/409,123, filed on Sep. 5, 2002; each of which applications is entitled “EPITOPE SEQUENCES.” The analogs can further be used in any of the various modes described in those applications. Epitope clusters, which can include or include the instant analogs, are disclosed and more fully defined in U.S. patent application Ser. No. 09/561,571, filed on Apr. 28, 2000, entitled “EPITOPE CLUSTERS.” Methodology for using and delivering the instant analogs is described in U.S. patent application Ser. No. 09/380,534 and U.S. Pat. No. 6,977,074 (issued Dec. 20, 2005) and in PCT Application No. PCT/US98/14289 (Pub. No. WO 99/02183A2), each entitled “A METHOD OF INDUCING A CTL RESPONSE.” Beneficial epitope selection principles for such immunotherapeutics are disclosed in U.S. patent application Ser. No. 09/560,465, filed on Apr. 28, 2000, Ser. No. 10/026,066 (Pub. No. 2003-0215425 A1), filed on Dec. 7, 2001, and Ser. No. 10/005,905 filed on Nov. 7, 2001, all entitled “EPITOPE SYNCHRONIZATION IN ANTIGEN PRESENTING CELLS,” U.S. Pat. No. 6,861,234 (issued 1 Mar. 2005; U.S. patent application Ser. No. 09/561,074), entitled “METHOD OF EPITOPE DISCOVERY”; U.S. patent application Ser. No. 09/561,571, filed Apr. 28, 2000, entitled “EPITOPE CLUSTERS”; U.S. patent application Ser. No. 10/094,699 (Pub. No. 2003-0046714 Al), filed Mar. 7, 2002, entitled “ANTI-NEOVASCULATURE PREPARATIONS FOR CANCER”; U.S. patent application Ser. No. 10/117,937 (Pub. No. 2003-0220239 A1) and PCTUS02/11101 (Pub. No. WO 02/081646A2), both filed on Apr. 4, 2002, and both entitled “EPITOPE SEQUENCES”; and U.S. patent application Ser. No. 10/657,022 and PCT Application No. PCT/US2003/027706 (Pub. No. WO 04/022709A2) both filed on Sep. 5, 2003, and both entitled “EPITOPE SEQUENCES.” Aspects of the overall design of vaccine plasmids are disclosed in U.S. patent application Ser. No. 09/561,572, filed on Apr. 28, 2000, entitled “EXPRESSION VECTORS ENCODING EPITOPES OF TARGET-ASSOCIATED ANTIGENS”; and Ser. No. 10/292,413 (Pub. No. 2003-0228634 A1), filed on Nov. 7, 2002, entitled “EXPRESSION VECTORS ENCODING EPITOPES OF TARGET-ASSOCIATED ANTIGENS AND METHODS FOR THEIR DESIGN”; U.S. patent application Ser. No. 10/225,568 (Pub No. 20030138808), filed on Aug. 20, 2002, PCT Application No. PCT/US2003/026231 (Pub. No. WO 2004/018666), filed on Aug. 19, 2003, both entitled “EXPRESSION VECTORS ENCODING EPITOPES OF TARGET-ASSOCIATED ANTIGENS”; and U.S. Pat. No. 6,709,844 entitled “AVOIDANCE OF UNDESIRABLE REPLICATION INTERMEDIATES IN PLASMID PROPAGATION.” Specific antigenic combinations of particular benefit in directing an immune response against particular cancers are disclosed in Provisional U.S. patent application Ser. No. 60/479,554, filed on Jun. 17, 2003 and U.S. patent application Ser. No. 10/871,708 (Pub. No. 20050118186), filed on Jun. 17, 2004 and PCT Patent Application No. PCT/US2004/019571 (Pub. No. WO 2004/112825), all entitled “COMBINATIONS OF TUMOR-ASSOCIATED ANTIGENS IN VACCINES FOR VARIOUS TYPES OF CANCERS.” Antigens associated with tumor neovasculature (e.g., PSMA, VEGFR2, Tie-2) are also useful in connection with cancerous diseases, as is disclosed in U.S. Pat. No. 7,252,824 (issued Aug. 7, 2007), U.S. patent application Ser Nos. 11/073,347 (Pub. No. 20050260234) and 11/772,811 (Pub. No. 20090208537), all entitled “ANTI-NEOVASCULATURE PREPARATIONS FOR CANCER,” each of which is hereby incorporated by reference in its respective entirety. Methods to trigger, maintain, and manipulate immune responses by targeted administration of biological response modifiers are disclosed U.S. Provisional Application No. 60/640,727, filed on Dec. 29, 2004. Methods to bypass CD4+ cells in the induction of an immune response are disclosed in U.S. Provisional Application No. 60/640,821, filed on Dec. 29, 2004. Exemplary diseases, organisms and antigens and epitopes associated with target organisms, cells and diseases are described in U.S. Pat. No. 6,977,074 (issued Dec. 20, 2005) filed Feb. 2, 2001 and entitled “METHOD OF INDUCING A CTL RESPONSE.” Exemplary methodology is found in U.S. Provisional Application No. 60/580,969, filed on Jun. 17, 2004, and U.S. patent application Ser. No. 20060008468-A1, published on Jan. 12, 2006, both entitled “COMBINATIONS OF TUMOR-ASSOCIATED ANTIGENS IN DIAGNOTISTICS FOR VARIOUS TYPES OF CANCERS.” Methodology and compositions are also disclosed in U.S. Provisional Application No. 60/640,598, filed on Dec. 29, 2004, entitled “COMBINATIONS OF TUMOR-ASSOCIATED ANTIGENS IN COMPOSITIONS FOR VARIOUS TYPES OF CANCER.” The integration of diagnostic techniques to assess and monitor immune responsiveness with methods of immunization including utilizing the instant analogs is discussed more fully in Provisional U.S. patent application Ser. No. 60/580,964 filed on Jun. 17, 2004 and U.S. patent application Ser. No. 2005-0287068-A1, published on Dec. 29, 2005) both entitled “IMPROVED EFFICACY OF ACTIVE IMMUNOTHERAPY BY INTEGRATING DIAGNOSTIC WITH THERAPEUTIC METHODS.” The immunogenic polypeptide encoding vectors are disclosed in U.S. patent application Ser. No. 10/292,413 (Pub. No. 2003-0228634 A1), filed on Nov. 7, 2002, entitled “EXPRESSION VECTORS ENCODING EPITOPES OF TARGET-ASSOCIATED ANTIGENS AND METHODS FOR THEIR DESIGN,” and in U.S. Provisional Application No. 60/691,579, filed on Jun. 17, 2005, entitled “METHODS AND COMPOSITIONS TO ELICIT MULTIVALENT IMMUNE RESPONSES AGAINST DOMINANT AND SUBDOMINANT EPITOPES, EXPRESSED ON CANCER CELLS AND TUMOR STROMA.” Additional useful disclosure, including methods and compositions of matter, is found in U.S. Provisional Application No. 60/691,581, filed on Jun. 17, 2005, entitled “MULTIVALENT ENTRAIN-AND-AMPLIFY IMMUNOTHERAPEUTICS FOR CARCINOMA.” Each of the applications and patents mentioned in the above paragraphs is hereby incorporated by reference in its entirety for all that it teaches. Additional analogs, peptides and methods are disclosed in U.S. Patent Publication No. 2006-0057673 A1, published on Mar. 16, 2006, entitled “EPITOPE ANALOGS,” and PCT Application Publication No. WO/2006/009920, entitled “EPITOPE ANALOGS,” all filed on Jun. 17, 2005. As an example, without being limited thereto each reference is incorporated by reference for what it teaches about class I MHC-restricted epitopes, analogs, the design of analogs, uses of epitopes and analogs, methods of using and making epitopes, and the design and use of nucleic acid vectors for their expression. Other applications that are expressly incorporated herein by reference are: U.S. patent application Ser. No. 11/321,967 (Pub. No. 2006-0153844), filed on Dec. 29, 2005, entitled “METHODS TO TRIGGER, MAINTAIN AND MANIPULATE IMMUNE RESPONSES BY TARGETED ADMINISTRATION OF BIOLOGICAL RESPONSE MODIFIERS INTO LYMPHOID ORGANS,” U.S. patent application Ser. No. 11/323,572 (Pub. No. 2006-0165711), filed on Dec. 29, 2005, entitled “METHODS TO ELICIT ENHANCE AND SUSTAIN IMMUNE REPONSES AGAINST MCH CLASS I RESTRICTED EPITOPES, FOR PROPHYLACTIC OR THERAPEUTIC PURPOSES,” U.S. patent application Ser. No. 11/323,520 (Pub. No. 2008-0124352), filed Dec. 29, 2005, entitled “METHODS TO BYPASS CD4+ CELLS IN THE INDUCTION OF AN IMMUNE RESPONSE,” U.S. patent application Ser. No. 11/323,049 (Pub. No. 2006-0159694), filed Dec. 29, 2005, entitled “COMBINATION OF TUMOR-ASSOCIATED ANTIGENS IN COMPOSITIONS FOR VARIOUS TYPES OF CANCERS,” U.S. patent application Ser. No. 11,323,964 (Pub. No. 2006-0159689), filed Dec. 29, 2005, entitled “COMBINATIONS OF TUMOR-ASSOCIATED ANTIGENS IN DIAGNOSTICS FOR VARIOUS TYPES OF CANCERS,” U.S. Provisional Application Ser. No. 60/691,889, filed on Jun. 17, 2005 entitled “EPITOPE ANALOGS.” Methods for enhancing a T cell response are disclosed in U.S. patent application Ser. No. 12/070,156 (Pub. No. 2008-0199485). Each of the foregoing disclosures is hereby incorporated by reference in its entirety.

The active immunotherapeutic described herein can be used in the manufacture of a medicament for use in the treatment of a disease, for example, a skin and/or lymphatic disease, a cancer, in a patient.

The following examples are for illustrative purposes only and are not intended to limit the scope of the disclosure or its various embodiments in any way.

### EXAMPLES

The following examples are included to demonstrate embodiments disclosed herein. It is appreciated by those of skill in the art that the methodology and compositions disclosed in the examples which follow represent methodology discovered by the inventors to function well in the practice of the disclosure, and thus can be considered to constitute particular modes for its practice. However, those of skill in the art can, in light of the present disclosure, appreciate that many changes can be made in the specific embodiments which are disclosed and still obtain a like or similar result without departing from the spirit and scope of the disclosure.

### Example 1

### Evaluation of Immunity and Clinical Outcome of an Active Immunotherapy in Patients with Advanced Melanoma

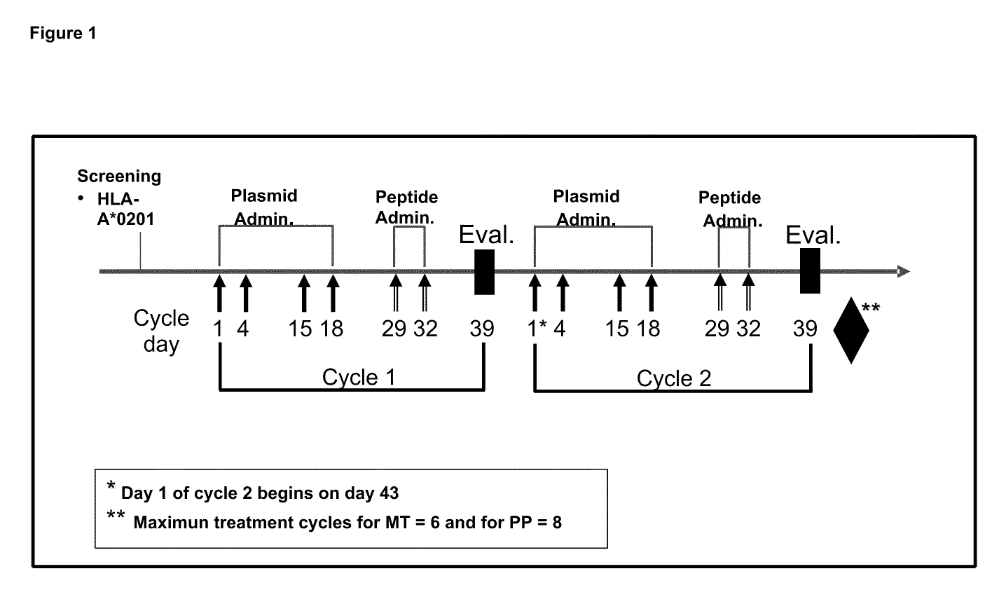

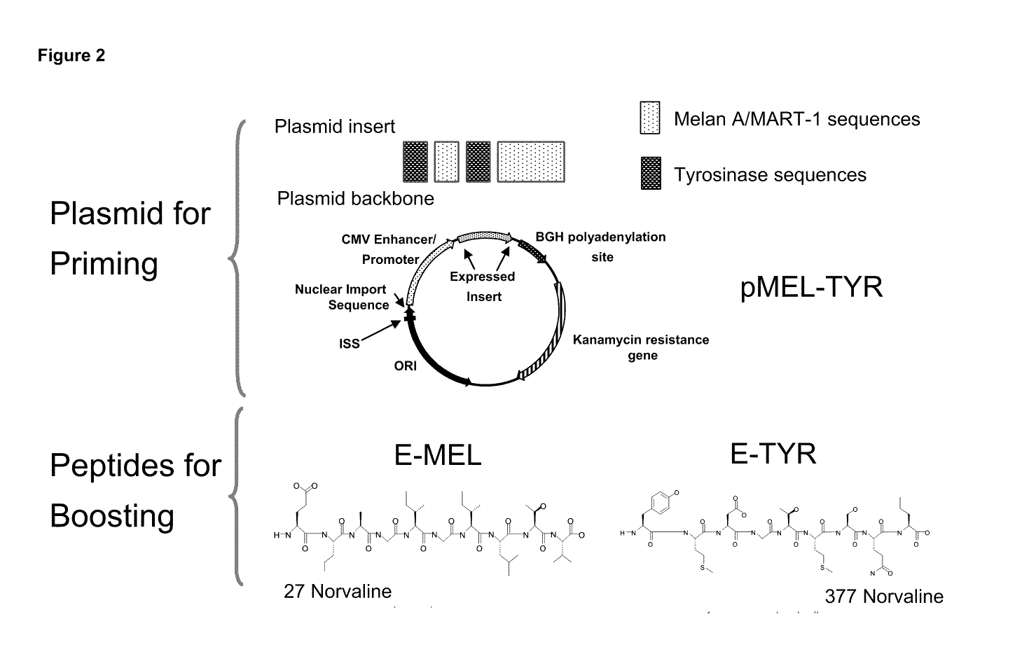

In an open-label, multicenter trial, an active immunotherapeutic product (referred to as MKC1106-MT) encompassing three components: a recombinant plasmid expressing fragments of two targeted antigens, Melan A/MART-1 and Tyrosinase (pMEL-TYR); and two peptides, epitope analogues corresponding to the respective target CTL epitopes Melan A/MART-1₂₆₋₃₅ and Tyrosinase₃₆₉₋₃₇₇ (E-MEL and E-TYR, respectively), was tested in an immunization regimen as shown in FIG. 1. The plasmid design and peptide analogues are as shown in FIG. 2. The plasmid was formulated as a sterile solution for administration at a concentration of 4.0 mg/mL. Each of the peptides, (E-MEL and E-TYR), were formulated as sterile solutions from lyophilized powders at a concentration of 1.0 mg/mL prior to injection.

All patients enrolled in this trial were HLA-A2 positive. These patients also had confirmed advanced (stage IIIb, Mc or IV) melanoma, progressing through prior therapies (for example, refractory to standard chemotherapy, radiation and/or surgery).

Using a treatment schedule/protocol as shown in FIG. 1, the multicomponent active immunotherapeutic product was delivered by intralymph node injection using a plasmid prime/peptide boost approach aimed to elicit a functional immune response against both the Melan A/MART-1 and tyrosinase antigens. The plasmid dose for bilateral administration into inguinal lymph nodes was fixed at 1,200 micrograms per injection. A total of eighteen patients were dosed—seven in the low dose peptide cohort and eleven in the high dose cohort. The low dose peptide cohort received a low dose' of 100 micrograms each of the Melan A/MART1 and tyrosinase peptides per injection. The high dose peptide cohort received a ‘high dose’ of 300 micrograms each of the Melan A/MART1 and tyrosinase peptides per injection.

In the first therapeutic cycle, plasmid-priming treatment was administered on days 1, 4, 15 and 18 followed by the peptide-boost treatment on days 29 and 32. This was followed by a second therapeutic cycle. Clinical evaluation occurred after each therapeutic cycle (one cycle=6 weeks). Patients who were non-progressors per protocol continued with additional treatment cycles until change in status (disease progression (PD)) and could receive up to 8 cycles of treatment, spanning 48 weeks. The immune response was assessed at baseline and throughout therapy by measuring the percentage of CD8+ T cells, in peripheral blood, specific for select epitopes of the two immunizing antigens. Clinical responses were categorized using RECIST (Response Evaluation Criteria in Solid Tumor) criteria. Patients were assessed as having stable disease (SD) if they maintained such status for a minimum of 8 weeks; or as applicable, a partial response (PR) or complete response (CR), based on measurable changes in tumor size confirmed by repeat assessment at 4 weeks.

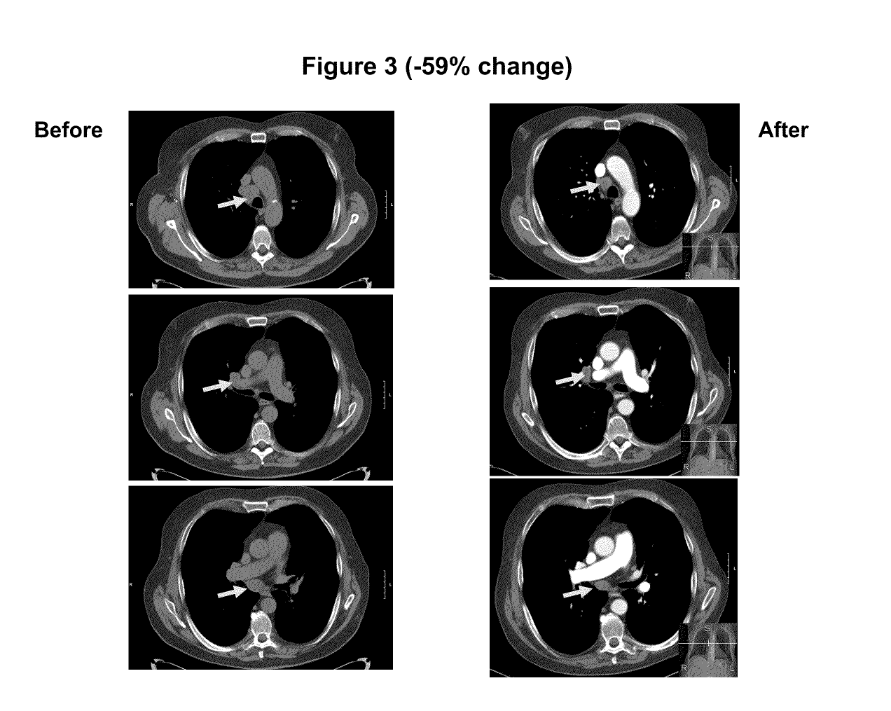

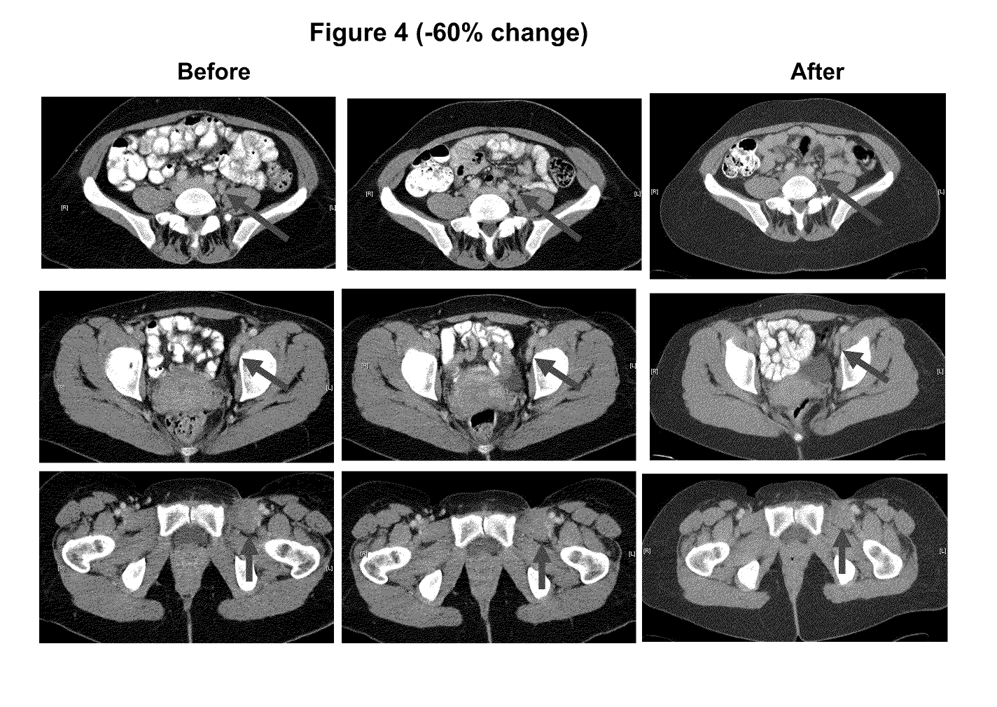

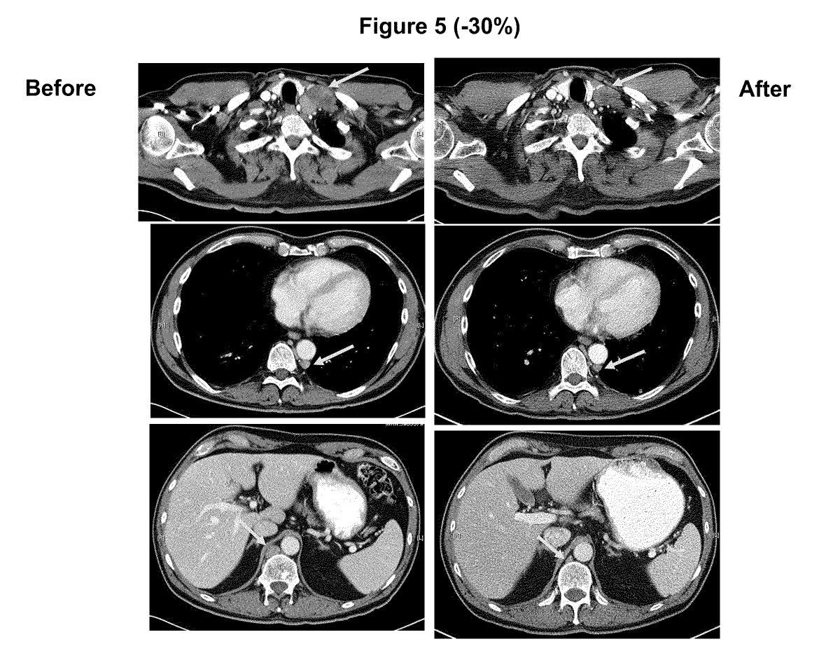

Ten patients completed two or more cycles of therapy (two patients in the low dose and eight in the high dose). All patients evaluated co-expressed the target antigens in tumor biopsies. Six patients (one patient in the low dose cohort and five in the high dose cohort) exhibited tumor response as defined by RECIST criteria. Of these six patients, three exhibited Partial Response (PR) and three exhibited Stable Disease (SD). Two of the three patients with PR completed eight cycles of therapy; and the third patient with PR completed an additional cycle for a total of nine cycles (=1 year) of therapy. These three patients with PR exhibited 59% (FIG. 3), 60% (FIG. 4) and 30% (FIG. 5) tumor shrinkage in combined targeted lesions. One of the three patients with SD completed 8 cycles of therapy and the other two patients progressed after two cycles of therapy.

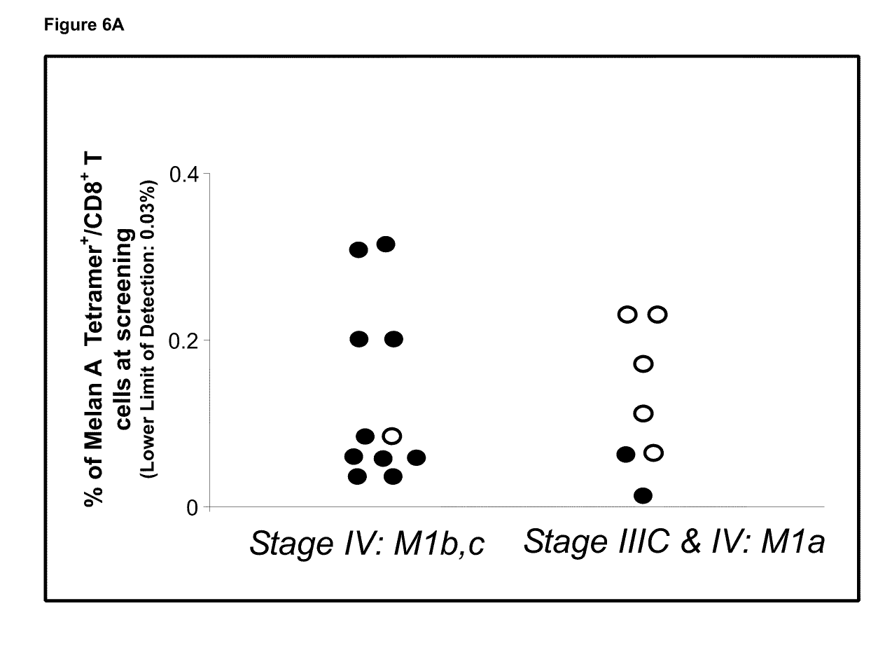

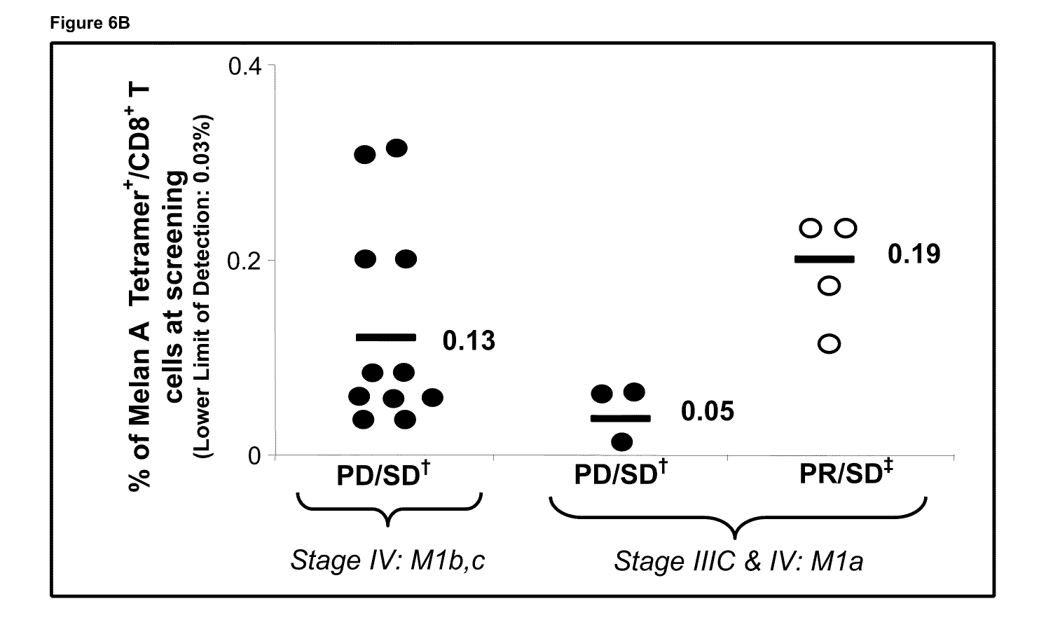

Five of these 6 patients with a tumor response were stage IIIC or IV with lymphatic metastasis (3 PRs and 2 SDs) (FIG. 6A; open circles; Stage IIIC & IV: M1a). Among those, four patients completed eight or nine cycles of therapy, all of whom had lymphatic disease and presence of Melan A specific T-cells at baseline (FIG. 6B open circles; Stage IIIC & IV: M1a; PR/SD). A fifth patient with stage IIIC lymphatic disease but without the presence of Melan A specific T cells at baseline, achieved SD at 2 cycles of therapy and later progressed without continuing on trial past 2 cycles (FIG. 6A; open circle; Stage IIIC & IV: M1a). The sixth patient with stage IV (M1b) achieved SD at two cycles of therapy and later progressed without continuing on trial past 2 cycles (FIG. 6A; open circle; Stage IV: M1b, M1c).

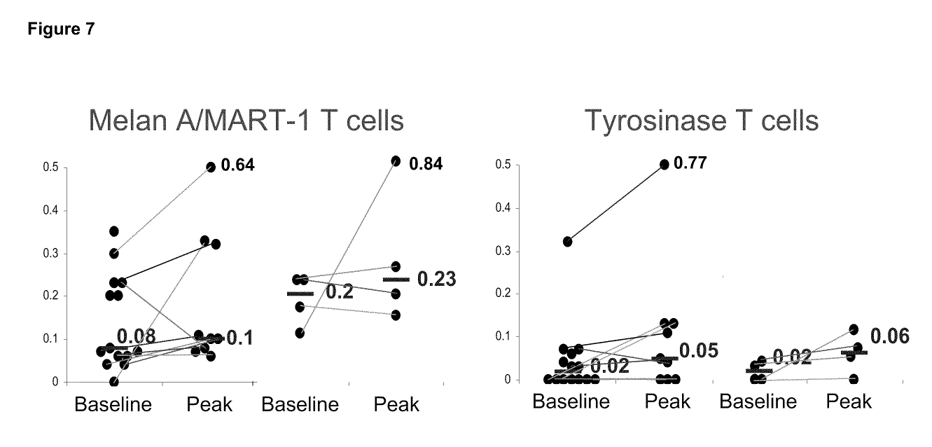

The results show that the immune response rate, defined as the percentage of patients who showed elevated numbers of antigen specific T cells in the blood upon immunization, as measured by MHC multimer staining, was achieved in 50% of all patients treated (FIG. 7). Among two biopsies of regressing lesions one had a dense infiltration of CD8+ T cells with about 1% TILs (tumor infiltrating lymphocytes) being specific for the Melan A epitope. In addition, the tumor biopsies showed heavy infiltration with both CD8+ and CD4+ T cells.

Repeated administration of the regimen was safe and well tolerated with all patients completing at least one cycle and four patients completing at least eight cycles (one year) of therapy. No drug-related serious adverse effects (SAEs) were reported. The most frequently reported adverse effects (AEs) were Grade 1 or 2 adverse events primarily involving pain in the injection area (unilateral and bilateral groin) and fatigue (intermittent), fever, lightheadedness, rash, pruritis, flushing and erythema.

Subset analysis identified patient populations in the trial—based on disease stage and baseline specific T cells—who demonstrated the best overall clinical responses. Among 8 patients who had Melan A/MART-1 specific T cells at baseline, four patients (all with lymphatic metastatic disease) achieved durable (≧1 year) clinical responses, (PR or SD as measurable by RECIST, and tumor regression), while the other 4 patients (all with visceral disease) showed rapid disease progression and did not complete 2 cycles of treatment. Among the remaining 10 patients without measurable specific T cells at baseline, none achieved durable responses.

Of the 7 patients with skin and/or lymphatic disease (stage IIIC and IV M1a), 4 showed durable clinical response. Further supporting the role of disease stage in clinical response, none of the 11 patients with visceral disease (M1b, M1c) had durable clinical responses. In those 4 patients with durable clinical responses, none of them had mixed responses in that no new lesions were developed in any of these 4 patients, suggesting activity against micrometastatic foci, since the natural progression of such patients is disseminated, metastatic disease. Taken together, these results not only support clinical applicability of this agent in a measurable disease setting (such as lymphatic metastatic disease), but also suggest its effectiveness against micrometastatic disease applicable to two indications: 1) adjuvant setting (for example after primary treatment in patients with minimal residual disease) to prolong time to relapse; 2) in advanced disease, adjunctive therapy or monotherapy, to slow down tumor progression by preventing development of new metastatic lesions.

### Example 2

### Evaluation of Immunity and Clinical Outcome of an Active Immunotherapy in Patients with PRAME and PSMA Positive Solid Tumors

Accumulating evidence on tissue expression and epitope immunogenicity indicate that PRAME (Preferential Antigen of Melanoma) and PSMA (Prostate Specific Membrane Antigen) are targets potentially applicable to active immunotherapy of diverse solid tumors.

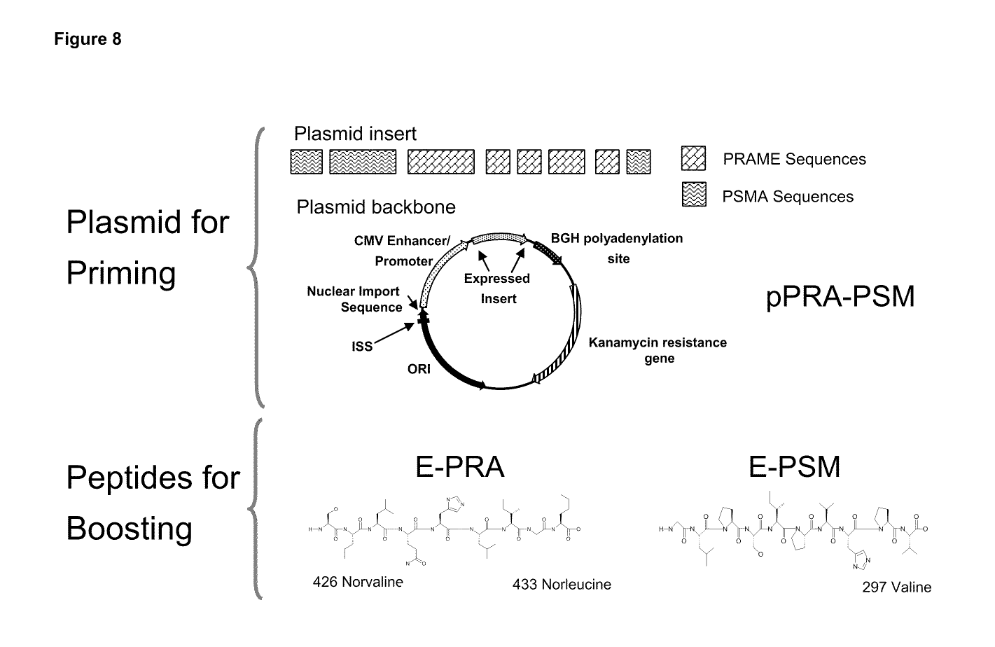

Therefore, in a second phase 1, open-label, multicenter study, HLA-A2 positive patients co-expressing PRAME and PSMA in tumor tissue, and with advanced cancer of diverse tumor types, metastatic disease and/or progressive, refractory disease were treated with an active immunotherapeutic product targeting PRAME and PSMA antigens. The active immunotherapeutic product (referred to as MKC1106-PP) encompassed three components: a recombinant plasmid (pPRA-PSM) expressing fragments of the two targeted antigens; and two peptide analogues (E-PRA and E-PSM), corresponding to the respective target antigens (FIG. 8). The plasmid was formulated as a sterile solution at a concentration of 4.0 mg/mL and each of the peptides, (E-PRA and E-PSM), were formulated as sterile solutions (at a concentration of 0.5 mg/mL and 1.0 mg/mL respectively) from lyophilized powders. Each of the three components of the immunotherapeutic regimen was delivered by bilateral intra-inguinal lymph node injection in a prime-boost protocol, aimed to elicit a functional immune response against both PRAME and PSMA antigens. The plasmid and peptide schedule of administration is shown in FIG. 1.

A total of twenty-six patients with various tumor types (11 prostate carcinoma, 2 kidney carcinoma, 11 melanoma, and 2 with other cancers) were dosed: thirteen each in the low and high dose peptide cohort, respectively. Twenty-four patients completed at least one cycle of treatment and were deemed to be evaluable for immune response. The two peptide doses used were: ‘low dose’ of 22.5 and 30 micrograms, and ‘high dose’ of 150 and 300 micrograms of peptide/injection, for E-PRA and E-PSM, respectively. The plasmid dose for bilateral injection was fixed at 1,200 micrograms/injection.

Subjects were evaluated clinically after two therapeutic cycles (12 weeks). Patients determined to be non-progressors (i.e. disease progression not observed; responders) continued on therapy and could receive up to 6 cycles of treatment (spanning 36 weeks), beyond two therapeutic cycles. The immune response was assessed by measuring the percentage of CD8+ T cells specific for the two immunizing antigens, PRAME and PSMA, in peripheral blood, at baseline and throughout therapy using tetramer and ELISPOT assays.

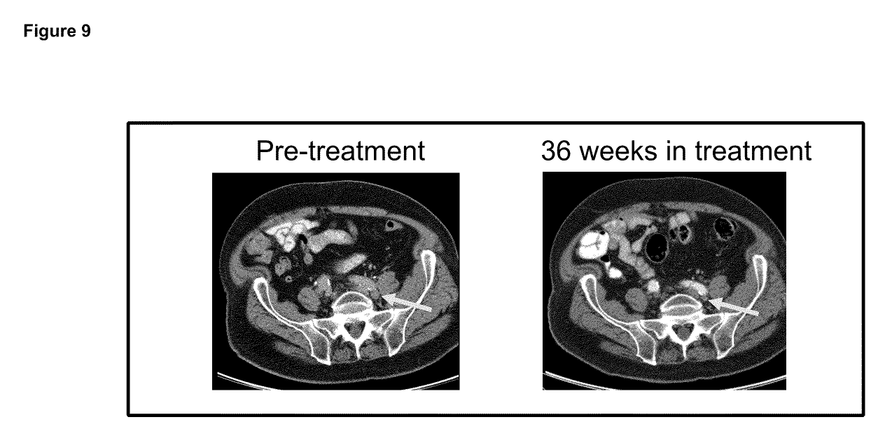

Seven patients showed emerging evidence of durable clinical response, completing four or more cycles of therapy during at least six months (3 patients in the low dose and 4 in the high dose): 4 out of 10 prostate carcinoma patients; both kidney cancer patients, and 1 out of 11 melanoma patients. FIG. 9 depicts tumor shrinkage in a prostate cancer patient with lymphatic metastatic disease at 36 weeks after treatment initiation. Fifteen out of twenty-four evaluable patients showed transient or persistent expansion of T cells specific to the immunizing antigens, subsequent to initiation of the immunization protocol.

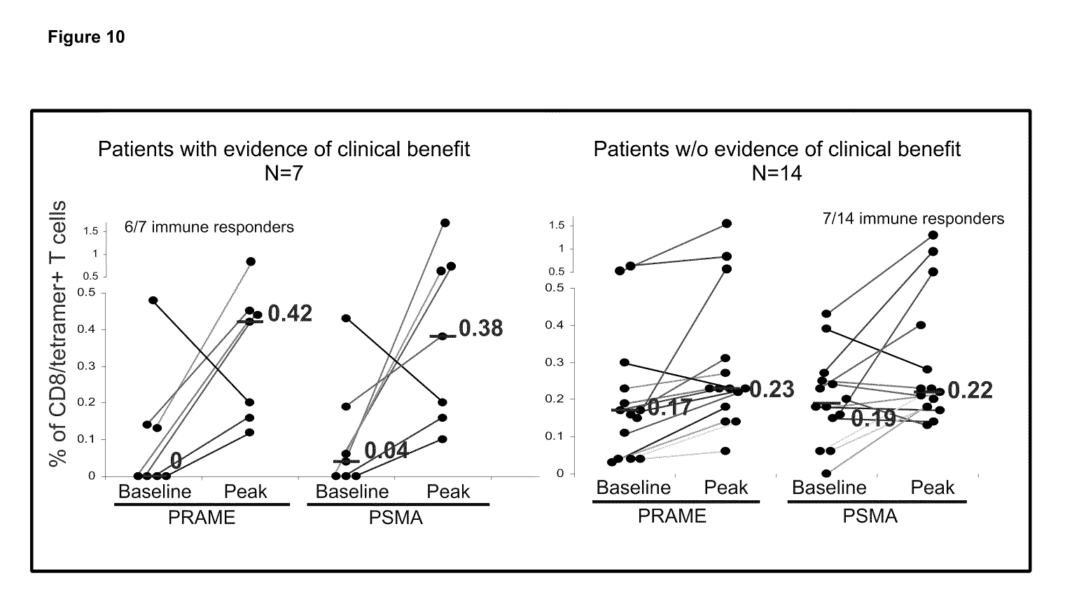

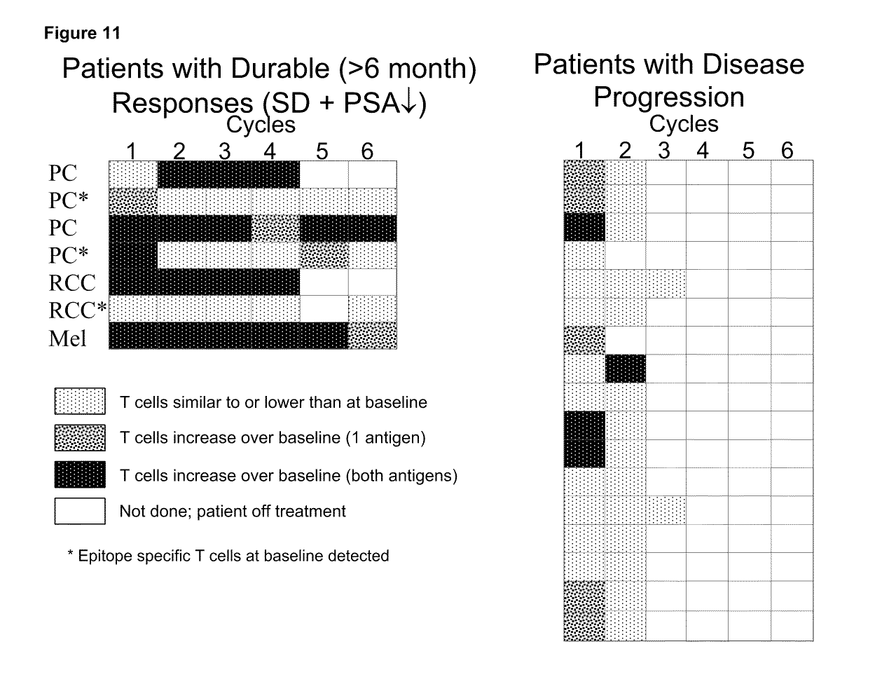

Clinical benefit was observed to be associated with significant expansion of antigen specific T cells as measured by tetramer analysis. In particular, patients who showed evidence of durable clinical response tended to show expansion of specific T cells in the blood early on and, in a larger proportion, after initiation of the regimen. Durable clinical benefit (SD over at least four cycles of treatment) was observed in 7 evaluable patients (FIG. 10 and Table 3) compared to 17 patients with disease progression (FIG. 10 and Table 3) by the time they had the first evaluation after treatment was initiated (12 weeks). Of the 17 patients with disease progression (Table 3), 14 patients had measurable T cells in the blood at initiation and during treatment (see FIG. 10; N=14); the other 3 patients disease progressed too rapidly to obtain measurable T cells in the blood beyond initiation of treatment. In addition, half of the patients evaluated who showed evidence of durable clinical response, also displayed persistent T cells in the blood throughout and beyond the first 2 cycles (12 weeks) compared to 0% (zero out of seventeen patients) who progressed within the same interval, corresponding to the first two cycles of therapy (FIG. 11; * Epitope specific T cells at baseline detected). Finally, patients who showed expansion of specific T cells and durable clinical response tended to display a lower percentage, or no antigen specific T cells at baseline, indicating a de novo induction of a T cell response with effector capabilities in patients with competent T cell immunity. In addition, the data suggest a pre-existing, impaired T cell response in patients with disease that progressed rapidly despite treatment.

TABLE 3
Association between immunity, disease stage and durable clinical benefit.

| | | | | | | | | # of pts who have |
|---|---|---|---|---|---|---|---|---|
| | | Median# | | | | | | expansion and |
| | | of Wks on | | | | Peak % T cells after | | persistence of |
| | # | treatment | # of | % T cells at baseline | | immunization | | T cells beyond |
| | of | Median | “Imm. | Median (range) | | Median (range) | | the first 2 cycles |
| Outcome | pts | (range) | Resp.” | PRAME | PSMA | PRAME | PSMA | PRAME & PSMA |
| Disease | 17 | 12 | 9/17 | 0.17 | 0.19 | 0.23 | 0.22 | 0/17 |
| progression | | (0-18) | | (0-0.62) | (0-0.43) | (0.08-1.54) | (0.13-1.29) | |
| Durable | 7 | 26 | 6/7 | 0.0 | 0.04 | 0.42 | 0.38 | 4/7 |
| clinical | | (24-36) | | (0-0.48) | (0-0.44) | (0.16-0.84) | (0.16-1.67) | |
| response | | | | | | | | |

Target expression analysis confirmed co-expression of PRAME and PSMA in nearly 100% of prostate cancer tumor samples and a majority of tumor tissues from kidney cancer and melanoma patients. The best clinical outcomes include: a prostate carcinoma patient with tumor regression mirrored by PSA decline (30+ weeks), two prostate carcinoma patients with stable disease (36+ weeks) accompanied by PSA velocity change, a kidney cancer patient with no evidence of disease post-resection in neoadjuvant setting (72+ weeks) and a metastatic melanoma patient (M1c stage) with stable disease at 72+ weeks.

Overall, the results show that the immune response rate, defined as the percentage of patients who showed elevated numbers of antigen specific T cells in the blood upon immunization, was greater than 60% among evaluable patients. The immunization regimen was well tolerated with five patients completing six cycles (nine months) of therapy. Seven patients achieved clinical response defined as ‘Partial Response’ (RECIST), or change in PSA doubling time or ‘Stable Disease’ (for at least six months): four with prostate, two with renal carcinoma and one with melanoma. Patients who mounted an immune response against both antigens, persisting throughout the first 2 cycles of therapy were more likely to show clinical benefit.

No drug-related serious adverse effects (SAEs) were reported. The most frequently reported adverse effects (AEs) were Grade 1 or 2 adverse events primarily involving pain in the injection area (unilateral and bilateral groin) and fatigue (intermittent), fever, lightheadedness, rash, pruritis, flushing and erythema.

In some embodiments, the numbers expressing quantities of ingredients, properties such as molecular weight, reaction conditions, and so forth used to describe and claim certain embodiments of the disclosure are to be understood as being modified in some instances by the term “about.” Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the disclosure are approximations, the numerical values set forth in the specific examples are reported as precisely as practicable. The numerical values presented in some embodiments of the disclosure may contain certain errors necessarily resulting from the standard deviation found in their respective testing measurements.

### Example 3

### Independent Review of the Imaging Data from the Melanoma Trial

Objective responses, particularly tumor shrinkage, have not generally been observed in trials of cancer vaccines. For example, the recent approval of PROVENGE was based on a modest 4.1-month increase in length of survival among vaccine recipients; use of the agent was not associated with measurable antitumor effect (D. Longo, N. Engl. J. Med. 363:479-481, 2010; P. W. Kantoff, N. Engl. J. Med. 363:411-422, 2010). Thus, the generation of objective responses in the trials described above was surprising, such an observation could not have been expected given the small number of patients enrolled. The unexpected nature of these observations led to having the results from the melanoma trial reviewed by an independent pathology lab. Interpretation of CT scans and other imaging will vary quantitatively based on methodology, including selection of lesions to be evaluated—thus the value of independent reads—however should result in consistent qualitative results. As such, it is not expected that each independent read will exactly agree in all details, however, overall conclusions are expected to be consistent. While it is required to have independent reading of imaging results in efficacy trials for cancer treatments, it is atypical for a phase 1 trial.

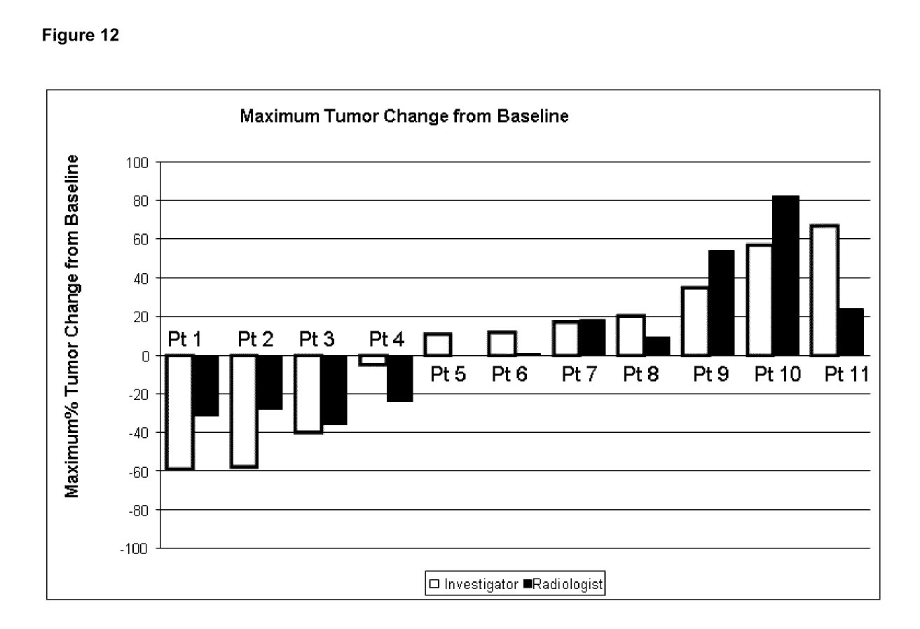

For the review, the independent radiologist was aware of tumor type and the time sequence of images but all other patient information, including treatment, was blinded. The independent radiologist was wholly responsible for selection of target and non-target lesions, their measurement, and assessing best response according to RECIST (v. 1.0) criteria based on digitized CT scans for all subjects from the trial for which imaging was available. (One responding subject had skin lesions only (Stage IIIC disease; see FIG. 6A and 6B) and thus no CT scans had been taken.) As seen in FIG. 12, a subject by subject comparison of maximum percent tumor change shows that the trial investigator and independent reads produced consistent results. Thus by this evaluation Pt-Pt4 and Pt6 (Pt 5 was not evaluable) showed objective response by RECIST criteria (the response for Pt6 were relatively short-lived) and Pt 7 to Pt11 had progressive disease despite treatment. Patients (Pt)1 to Pt4 in FIG. 12 were those identified as having pre-existing immunity to Melan A and classified as having Stage IV:M 1 a lymphatic disease (see FIG. 6A and 6B). Those patients classified as having Stage IV: M1b or M1c disease in FIG. 6A and 6B for having visceral target lesions include Pt 5 to Pt 11 in FIG. 12.

The independent read also reported some lesions outside the lymphatic system for Pt1 to Pt4, including lesions in lung, liver, and soft tissue, not reported by the trial investigator. These lesions did not change in size or number over the course of treatment and were generally less than 1 cm in their largest dimension (non-target lesions). These observations indicated that patients with predominant lymphatic disease but otherwise low visceral tumor burden can benefit from this immunotherapy. Moreover, they were consistent with the idea that further metastasis can be inhibited by this treatment.

In some embodiments, the terms “a,” “an” and “the” and similar referents used in the context of describing a particular embodiment of the disclosure (especially in the context of certain of the following claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Particular embodiments of this disclosure are described herein, including the best mode known to the inventors in the practice of the disclosure. Variations on those embodiments will become apparent to those of ordinary skill in the art upon reading the foregoing description. It is contemplated that skilled artisans may employ such variations as appropriate, and the disclosure may be practiced otherwise than specifically described herein. Accordingly, many embodiments of this disclosure include all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, numerous references have been made to patents and printed publications throughout this specification. Each of the above cited references and printed publications is herein individually incorporated by reference in its entirety to the extent that their content does not conflict with the disclosure directly present herein.

The foregoing paragraphs are intended to illustrate how the general concepts of the disclosure can be applied in practice and are not to be taken as an exhaustive or limiting recitation of the possible variations. Indeed many further variations or modifications will be suggested by the properties of particular immunotherapeutic compositions and immunization regimens/protocols, and will become apparent to those of skill in the art. Thus, by way of example, but not of limitation, alternative configurations of the present disclosure may be utilized in accordance with the teachings herein. Individual embodiments can specifically include or exclude any such alternatives. Accordingly, the present disclosure is not limited to that precisely as shown and described.

1. A method of treating a cancer patient with an active immunotherapeutic comprising
assessing a patient's level of pre-existing immunoreactivity to at least one target antigen;
choosing an immunotherapeutic regimen based on the level of pre-existing immunoreactivity, wherein the regimen comprises administration of at least one immunogen targeting the at least one target antigen, or an epitope thereof; and
treating the patient according to the regimen.

2. The method of claim 1, wherein said immunogen is capable of promoting a T cell effector response to an antigen associated with the cancer.

3. The method of claim 1, wherein the immunotherapeutic regimen achieves a clinical benefit.

4. The method of claim 3, wherein the clinical benefit comprises tumor regression or stabilization of disease.

5. The method of claim 1, wherein the level of pre-existing immunoreactivity is measured by tetramer or ELISPOT assay.

6. The method of claim 1, wherein the patient has a cancer that has not progressed beyond secondary lymphatic organs.

7. The method of claim 1, wherein the patient has stage IIIC or IV (M1a) lymphatic disease.

8. The method of claim 1, wherein the cancer is at least one of melanoma, kidney, breast, pancreas, prostate, colorectal, ovarian, non-small-cell-lung, glioblastoma, ocular melanoma, hormone sensitive carcinoma of breast, prostate, and ovary, hormone refractory prostate carcinoma, renal cell carcinoma, esophageal, or mesothelioma.

9. The method of claim 1, wherein the immunotherapeutic regimen comprises a prime-boost regimen.

10. The method of claim 1, wherein the immunotherapeutic regimen comprises more than one therapeutic cycle.

11. The method of claim 1, wherein administering the immunogen comprises direct delivery to the lymphatic system of the patient.

12. The method of claim 11, wherein the direct delivery to the lymphatic system comprises intranodal delivery.

13. The method of claim 1, wherein the patient is HLA-A2 positive.

14. The method of claim 1, wherein the target antigen comprises Melan A and the patient has a pre-existing immunoreactivity to Melan A, and wherein the immunotherapeutic regimen comprises administering an immunogen to promote an effector T cell response to Melan A, or an epitope thereof.

15. The method of claim 14, wherein the patient has a skin and/or lymphatic disease.

16. The method of claim 15, wherein the skin and/or lymphatic disease is stage IIIC or IV (M1a) lymphatic disease.

17. The method of claim 16, wherein the immunotherapeutic regimen comprises targeting Melan A₂₆₋₃₅ epitope or an analogue thereof.

18. The method of claim 14, wherein the cancer is melanoma or glioblastoma.

19. The method of claim 1, wherein the target antigen comprises at least one of PRAME or PSMA and the patient has no or minimal pre-existing immunoreactivity to at least one of PRAME or PSMA, and wherein the immunotherapeutic regimen comprises administering the immunogen to promote an effector T cell response to the at least one of PRAME or PSMA.

20. The method of claim 19, wherein PRAME and PSMA are co-expressed in the patient's tumor tissue.

21. The method of claim 19, wherein the immunotherapeutic regimen comprises targeting at least one of a PRAME₄₂₅₋₄₃₃ epitope or a PSMA₂₈₈₋₂₉₇ epitope.

22. The method of claim 19, wherein the cancer is prostate, kidney cancer, or melanoma.

23. The method of claim 19, wherein the cancer is prostate cancer and the clinical benefit comprises decreased PSA levels.

24. A method of treating a cancer patient comprising:
selecting a patient at a stage of disease wherein there is limited spread beyond the lymphatic system and any metastases that have spread beyond the lymphatic system number 10 or fewer and are either 1) not in a vital organ or 2) less than one centimeter in diameter; and
applying to the patient an immunotherapeutic regimen comprising administering directly to the lymphatic system of the patient an immunogen to promote a T cell effector response to an antigen associated with the cancer.


SEQUENCE LISTING (65 sequences)

SEQ ID NO: 1 - protein, 10 aa, Homo sapiens
EAAGIGILTV

SEQ ID NO: 2 - protein, 10 aa, Artificial Sequence
  T cell peptide epitope analogue
EXAGIGILTV

SEQ ID NO: 3 - protein, 10 aa, Artificial Sequence
  T cell peptide epitope analogue
ELAGIGILTV

SEQ ID NO: 4 - protein, 9 aa, Homo sapiens
YMDGTMSQV

SEQ ID NO: 5 - protein, 9 aa, Artificial Sequence
  T cell peptide epitope analogue
YMDGTMSQX

SEQ ID NO: 6 - protein, 9 aa, Homo sapiens
SLLQHLIGL

SEQ ID NO: 7 - protein, 9 aa, Artificial Sequence
  T cell peptide epitope analogue
SXLQHLIGX

SEQ ID NO: 8 - protein, 10 aa, Homo sapiens
GLPSIPVHPI

SEQ ID NO: 9 - protein, 10 aa, Artificial Sequence
  T cell peptide epitope analogue
GLPSIPVHPV

SEQ ID NO: 10 - protein, 10 aa, Artificial Sequence
  T cell peptide epitope analogue
EXAGIGILTX

SEQ ID NO: 11 - protein, 9 aa, Artificial Sequence
  T cell peptide epitope analogue
YXDGTMSQX

SEQ ID NO: 12 - protein, 10 aa, Artificial Sequence
  T cell peptide epitope analogue
EXAGIGILTV

SEQ ID NO: 13 - protein, 10 aa, Artificial Sequence
  T cell peptide epitope analogue
ELAGIGILTX

SEQ ID NO: 14 - protein, 9 aa, Artificial Sequence
  T cell peptide epitope analogue
YVDGTMSQX

SEQ ID NO: 15 - protein, 9 aa, Artificial Sequence
  T cell peptide epitope analogue
YVDGTMSQV

SEQ ID NO: 16 - protein, 9 aa, Artificial Sequence
  T cell peptide epitope analogue
XLLQHLIGL

SEQ ID NO: 17 - protein, 9 aa, Artificial Sequence
  T cell peptide epitope analogue
SXLQHLIGL

SEQ ID NO: 18 - protein, 9 aa, Artificial Sequence
  T cell peptide epitope analogue
SLXQHLIGL

SEQ ID NO: 19 - protein, 9 aa, Artificial Sequence
  T cell peptide epitope analogue
SLLQHXIGL

SEQ ID NO: 20 - protein, 9 aa, Artificial Sequence
  T cell peptide epitope analogue
SLLQHLIXL

SEQ ID NO: 21 - protein, 9 aa, Artificial Sequence
  T cell peptide epitope analogue
SLLQHLIGX

SEQ ID NO: 22 - protein, 9 aa, Artificial Sequence
  T cell peptide epitope analogue
XXLQHLIGL

SEQ ID NO: 23 - protein, 9 aa, Artificial Sequence
  T cell peptide epitope analogue
SXLQHLIGX

SEQ ID NO: 24 - protein, 9 aa, Artificial Sequence
  T cell peptide epitope analogue
XLLQHLIGV

SEQ ID NO: 25 - protein, 9 aa, Artificial Sequence
  T cell peptide epitope analogue
XLLQHLIGX

SEQ ID NO: 26 - protein, 9 aa, Artificial Sequence
  T cell peptide epitope analogue
XXLQHLIGX

SEQ ID NO: 27 - protein, 9 aa, Artificial Sequence
  T cell peptide epitope analogue
XLLQHLIGL

SEQ ID NO: 28 - protein, 9 aa, Artificial Sequence
  T cell peptide epitope analogue
SXLQHLIGL

SEQ ID NO: 29 - protein, 9 aa, Artificial Sequence
  T cell peptide epitope analogue
SLLQHLIGX

SEQ ID NO: 30 - protein, 9 aa, Artificial Sequence
  T cell peptide epitope analogue
SLLQHXIGL

SEQ ID NO: 31 - protein, 9 aa, Artificial Sequence
  T cell peptide epitope analogue
SXLQHLIGX

SEQ ID NO: 32 - protein, 9 aa, Artificial Sequence
  T cell peptide epitope analogue
XXLQHLIGX

SEQ ID NO: 33 - protein, 9 aa, Artificial Sequence
  T cell peptide epitope analogue
SXLQHLIGX

SEQ ID NO: 34 - protein, 9 aa, Artificial Sequence
  T cell peptide epitope analogue
TXLQHLIGX

SEQ ID NO: 35 - protein, 10 aa, Artificial Sequence
  T cell peptide epitope analogue
XLPSIPVHPI

SEQ ID NO: 36 - protein, 10 aa, Artificial Sequence
  T cell peptide epitope analogue
GXPSIPVHPI

SEQ ID NO: 37 - protein, 10 aa, Artificial Sequence
  T cell peptide epitope analogue
GXWSIPVHPI

SEQ ID NO: 38 - protein, 10 aa, Artificial Sequence
  T cell peptide epitope analogue
GLWSIPVHPX

SEQ ID NO: 39 - protein, 10 aa, Artificial Sequence
  T cell peptide epitope analogue
GLPSIPVHXI

SEQ ID NO: 40 - protein, 10 aa, Artificial Sequence
  T cell peptide epitope analogue
GLPSIPVHPX

SEQ ID NO: 41 - protein, 10 aa, Artificial Sequence
  T cell peptide epitope analogue
GXPSIPVHPX

SEQ ID NO: 42 - protein, 10 aa, Artificial Sequence
  T cell peptide epitope analogue
GXPSIPVHPX

SEQ ID NO: 43 - protein, 10 aa, Artificial Sequence
  T cell peptide epitope analogue
GXPSIPVHPV

SEQ ID NO: 44 - protein, 10 aa, Artificial Sequence
  T cell peptide epitope analogue
XLPSIPVHPX

SEQ ID NO: 45 - protein, 10 aa, Artificial Sequence
  T cell peptide epitope analogue
AXWSIPVHPI

SEQ ID NO: 46 - protein, 10 aa, Artificial Sequence
  T cell peptide epitope analogue
AVPSIPVHPX

SEQ ID NO: 47 - protein, 10 aa, Artificial Sequence
  T cell peptide epitope analogue
AXPSIPVHPV

SEQ ID NO: 48 - protein, 10 aa, Artificial Sequence
  T cell peptide epitope analogue
ALWSIPVHPX

SEQ ID NO: 49 - protein, 10 aa, Artificial Sequence
  T cell peptide epitope analogue
GVWSIPVHPX

SEQ ID NO: 50 - protein, 10 aa, Artificial Sequence
  T cell peptide epitope analogue
GXWSIPVHPV

SEQ ID NO: 51 - protein, 10 aa, Artificial Sequence
  T cell peptide epitope analogue
XLPSIPVHPI

SEQ ID NO: 52 - protein, 10 aa, Artificial Sequence
  T cell peptide epitope analogue
GXPSIPVHPI

SEQ ID NO: 53 - protein, 10 aa, Artificial Sequence
  T cell peptide epitope analogue
GLPSIPVHPX

SEQ ID NO: 54 - protein, 10 aa, Artificial Sequence
  T cell peptide epitope analogue
GLWSIPVHPX

SEQ ID NO: 55 - protein, 10 aa, Artificial Sequence
  T cell peptide epitope analogue
GXPSIPVHPX

SEQ ID NO: 56 - protein, 10 aa, Artificial Sequence
  T cell peptide epitope analogue
GXPSIPVHPV

SEQ ID NO: 57 - protein, 10 aa, Artificial Sequence
  T cell peptide epitope analogue
XLPSIPVHPX

SEQ ID NO: 58 - protein, 11 aa, Artificial Sequence
  T cell peptide epitope analogue
GXWPSIPVHPX

SEQ ID NO: 59 - protein, 10 aa, Artificial Sequence
  T cell peptide epitope analogue
AXWSIPVHPI

SEQ ID NO: 60 - protein, 10 aa, Artificial Sequence
  T cell peptide epitope analogue
AXPSIPVHPV

SEQ ID NO: 61 - protein, 10 aa, Artificial Sequence
  T cell peptide epitope analogue
GLWSIPVHPI

SEQ ID NO: 62 - protein, 62 aa, Artificial Sequence
  Synthetic peptide
KRSLLQHLIGLGDAAYSLLQHLIGLISPEKEEQYIASLLQHLIGLKRPSIKRGLPSIPVHPV

SEQ ID NO: 63 - protein, 275 aa, Artificial Sequence
  Synthetic peptide
MNLLHETDSAVATARRPRWLCAGALVLAGGFFLLGFLFGWFIKSAQLAGAKGVILYSDPADYFAPGVKSYPDGWNLPGGGVQRGNILNLNGAGDPLTPGYPANEYAYRRGIAEAVGLPSIPVHPIALQSLLQHLIGLSNLTHVLYPVPLESYEDIHGTLHLERLAYLHARLRELLCELGRPSMVWLSANPCPHCGDRTFYDPEPILCPCFMPNKRSLLQHLIGLGDAAYSLLQHLIGLISPEKEEQYIASLLQHLIGLKRPSIKRGLPSIPVHPV

SEQ ID NO: 64 - protein, 28 aa, Artificial Sequence
  Synthetic peptide
MLLAVLYCLELAGIGILTVYMDGTMSQV

SEQ ID NO: 65 - protein, 94 aa, Artificial Sequence
  Synthetic peptide
MLLAVLYCLELAGIGILTVYMDGTMSQVGILTVILGVLLLIGCWYCRRRNGYRALMDKSLHVGTQCALTRRCPQEGFDHRDSKVSLQEKNCEPV